SEQUENCE LISTING (2 sequences)

SEQ ID NO: 1 - DNA, 23 bp, Artificial Sequence
  Synthetic construct
cgctcaacactaugtgacctgcc

SEQ ID NO: 2 - DNA, 53 bp, Artificial Sequence
  Synthetic construct
tctatccgcctattcggcaggtcacauagtgttgagcggggagagctctagag

(19) United States
(12) Patent Application Publication
(10) Pub. No.: US 2025/0333434 A1
(43) Pub. Date: Oct. 30, 2025

(54) METHODS OF PREPARING BIVALENT MOLECULES

(71) Applicant: Insitro, Inc., South San Francisco, CA (US)

(72) Inventor: Richard Edward WATTS, Walnut Creek, CA (US)

(73) Assignee: Insitro, Inc., South San Francisco, CA (US)

(21) Appl. No.: 18/570,419

(22) PCT Filed: Jun. 16, 2022
(86) PCT No.: PCT/US2022/072994

§ 371 Date: Dec. 14, 2023

Related U.S. Application Data
(60) Provisional application No. 63/212,023, filed on Jun. 17, 2021.

(51) Int. Cl. C07H 21/00

(52) U.S. Cl. CPC C07H 21/00

(57) ABSTRACT

The present disclosure relates to bivalent or polyvalent linear initiator nucleic acids comprising initial building blocks and a coding region. The linear initiator nucleic acids may be used for the synthesis of an encoded compound to produce bivalent or polyvalent molecules.

### CROSS-REFERENCE TO RELATED APPLICATIONS

This application claims priority benefit to U.S. Provisional Application No. 63/212,023, filed on Jun. 17, 2021, titled “METHODS OF PREPARING BIVALENT MOLECULES”, the contents of which are incorporated herein by reference for all purposes.

### FIELD OF THE INVENTION

The present disclosure relates in some aspects to linear initiator nucleic acids, and methods of preparing thereof. The present disclosure also relates to methods of synthesizing compounds from linear initiator nucleic acids and methods to identify encoded molecules with desired properties using synthesized compounds.

### BACKGROUND OF THE INVENTION

The field of combinatorial chemistry has made it possible to prepare a large number of compounds in a single process. These combinatorial libraries are synthesized from successive chemical subunits (e.g., building blocks) that can be assembled on nucleic acids encoding the addition of these chemical subunits. The resulting library compounds may be tested for possession of desired properties (including, but not limited to, binding to a target molecule). Despite the success of many of these methods, existing methods have difficulty detecting interactions between library compounds and target molecules when the interactions are present in low numbers, or when the library compounds themselves are present in low numbers. Thus, there is a need in the art for improved libraries of compounds, including those that increase the number of interactions between library compounds and target molecules. Further, there is also a need for a method of synthesizing improved library compounds.

### SUMMARY OF THE INVENTION

Described herein are methods of preparing linear initiator nucleic acids. In some embodiments, there is a method of making a linear initiator nucleic acid, wherein the linear initiator nucleic acid comprises a first initial building block, a second initial building block, and a coding region; wherein the first initial building block is attached to a first site that is upstream of the coding region on the linear initiator nucleic acid and the second initial building block is attached to a second site that is downstream of the coding region on the linear initiator nucleic acid; the method comprising cleavage of a circularized nucleic acid to form the linear initiator nucleic acid; wherein the circularized nucleic acid comprises (i) the first initial building block, (ii) a cleavable linker, (iii) the second initial building block, and (iv) the coding region; wherein (i) and (iii) are attached to opposite ends of (ii), and wherein the cleavage cleaves the cleavable linker.

In some embodiments, the cleavage is by enzymatic cleavage. In some embodiments, the enzymatic cleavage is by restriction digestion. In some embodiments, the cleavage is by chemical cleavage.

In some embodiments, the circularized nucleic acid is formed by a method comprising ligation of a linear precursor nucleic acid to form the circularized nucleic acid; wherein the linear precursor nucleic acid comprises (i) the first initial building block, (ii) the cleavable linker, (iii) the second initial building block, and (iv) the coding region; wherein (i) and (iii) are attached to opposite ends of (ii) in the linear precursor nucleic acid; wherein (i), (ii), and (iii) are each upstream or each downstream of (iv) in the linear precursor nucleic acid. In some embodiments, the ligation is splint ligation. In some embodiments, the ligation is blunt ligation.

In some embodiments, the coding region comprises a plurality of codons. In some embodiments, at least one codon of the plurality of codons comprises from 5 to 60 nucleotides. In some embodiments, at least one codon encodes the addition of a polymer building block to the first initial building block, the second initial building block, or both. In some embodiments, the plurality of codons encodes for the addition of a plurality of polymer building blocks.

In some embodiments, the linear initiator nucleic acid comprises a first linker and a second linker, wherein the first linker attaches the first initial building block to the linear initiator nucleic acid, and the second linker attaches the second initial building block to the linear initiator nucleic acid. In some embodiments, the first initial building block is attached to the first linker by a covalent bond, and wherein the second initial building block is attached to the second linker by a covalent bond. In some embodiments, the first initial building block and the second initial building block are not nucleic acids or nucleic acid analogs.

In some embodiments, the coding region comprises from 2 to 20 codons. In some embodiments, the coding region comprises from 5 to 20 codons.

In some embodiments, the cleavable linker is an intervening sequence. In some embodiments, the intervening sequence is from 4 to 30 nucleotides long. In some embodiments, the intervening sequence is a non-nucleotide moiety.

Further described herein is a linear precursor nucleic acid comprising (i) a first initial building block, (ii) a cleavable linker, (iii) a second initial building block, and (iv) a coding region, wherein (i) and (iii) are attached to opposite ends of (ii) in the linear precursor oligonucleotide; wherein (i), (ii), and (iii) are each upstream or each downstream of (iv) in the linear precursor nucleic acid. In some embodiments of the linear precursor nucleic acid, the 5′ and 3′ termini are non-covalently bound to a nucleotide splint.

Further described herein is a circularized nucleic acid comprising (i) a first initial building block, (ii) a cleavable linker, (iii) a second initial building block, and (iv) a coding region; wherein (i) and (iii) are attached to opposite ends of (ii).

Further described herein is a method of synthesizing a compound comprising: (a) providing a pool of molecules comprising a plurality of linear initiator nucleic acids, wherein each linear initiator nucleic acid comprises a first initial building block, a second initial building block, and a coding region comprising a plurality of codons; wherein the first initial building block is attached to a site that is upstream of the coding region on the linear initiator nucleic acid and the second initial building block is attached to a second site that is downstream of the coding region on the linear initiator nucleic acid; (b) contacting at least one of the linear initiator nucleic acids with an anti-codon comprising a polymer building block under conditions which allow for hybridization of the anti-codon with at least one of the codons of the coding region, wherein the polymer building block reacts with the first initial building block or the second initial building block to form a covalent bond.

In some embodiments of a method of synthesizing a compound, the linear initiator nucleic acid is formed by cleavage of a cleavable linker at a cleavage site in a circularized nucleic acid comprising (i) the first initial building block, (ii) the cleavable linker comprising the cleavage site, (iii) a second initial building block, and (iv) the coding region; wherein (i) and (iii) are attached to opposite ends of (ii) in the circularized nucleic acid.

In some embodiments of a method of synthesizing a compound, the linear initiator nucleic acid comprises at the 5′ end a first portion of an intervening sequence and at the 3′ end a second portion of an intervening sequence; wherein the linear initiator nucleic acid was formed by restriction digestion of a restriction site in a circularized nucleic acid comprising (i) the first initial building block, (ii) the intervening sequence comprising the restriction site, (iii) a second initial building block, and (iv) the coding region; wherein (i) and (iii) are attached to opposite ends of (ii) in the circularized nucleic acid.

In some embodiments of a method of synthesizing a compound, the linear initiator nucleic acid comprises at the 5′ end a first portion of a cleavable linker and at the 3′ end a second portion of a cleavable linker; wherein the linear initiator nucleic acid was formed by restriction digestion of a restriction site in a circularized nucleic acid comprising (i) the first initial building block, (ii) the cleavable linker comprising the restriction site, (iii) a second initial building block, and (iv) the coding region; wherein (i) and (iii) are attached to opposite ends of (ii) in the circularized nucleic acid.

In some embodiments of a method of synthesizing a compound, the linear initiator nucleic acid may be prepared according to any of the methods described herein.

In some embodiments of a method of synthesizing a compound, the method further comprises repeating step (b) to form a synthesized compound comprising a plurality of polymer building blocks extending from the first initial building block and a synthesized compound comprising a plurality of polymer building blocks extending from the second initial building block. In some embodiments of a method of synthesizing a compound, the synthesized compound comprising the first initial building block and the synthesized compound comprising the second initial building block are the same.

In some embodiments of a method of synthesizing a compound, the polymer building block is not a nucleic acid or nucleic acid analog. In some embodiments of a method of synthesizing a compound, the synthesized compound does not comprise a nucleic acid or nucleic acid analog.

### BRIEF DESCRIPTION OF THE DRAWINGS

Representative embodiments of the invention are disclosed by reference to the following figures. It should be understood that the embodiments depicted are not limited to the precise details shown.

FIG. 1 shows a linear initiator nucleic acid comprising a first initial building block, a second initial building block, and a coding region comprising a plurality of codons, as prepared by the methods described herein. The first initial building block and the second initial building block are attached to the linear initiator nucleic acid via a linker.

FIG. 2 shows a linear precursor nucleic acid comprising a first initial building block, a second initial building block, a coding region comprising a plurality of codons, and a cleavable linker. The first initial building block and the second initial building block are attached at a position upstream and a position downstream of the cleavable linker, respectively. The first initial building block, the second initial building block, and the cleavable linker are each upstream of the coding region.

FIG. 3A shows a linear precursor nucleic acid splinted for ligation, to form a non-covalently circularized nucleic acid. The 5′ and 3′ termini of the linear precursor nucleic acid are non-covalently bound to a nucleotide splint.

FIG. 3B shows the orientation of a linear precursor nucleic acid prior to blunt end ligation. The linear precursor nucleic acid forms a circularized nucleic acid upon blunt end ligation.

FIG. 4 shows a circularized nucleic acid comprising a first initial building block, a second initial building block, a coding region, and a cleavable linker. The first initial building block and the second initial building block are attached at a position upstream and a position downstream of the cleavable linker, respectively.

FIG. 5 shows a cleavage site within a cleavable linker of a circularized nucleic acid with an attached splint that was used for ligation. The cleavage site may be a restriction site (an exemplary restriction site is shown) comprising a recognition sequence that is cleaved by restriction enzymes (e.g., at the dotted line), in order to produce a linear initiator nucleic acid. Alternatively, the cleavage site may be cleaved by chemical cleavage to produce a linear initiator nucleic acid.

FIG. 6 shows an exemplary method of making a linear initiator nucleic acid from a linear precursor nucleic acid with an intermediate circularized nucleic acid as described herein.

FIG. 7 shows a method of synthesizing a compound from a linear initiator nucleic acid and an anti-codon as described herein. The anti-codon carries a polymer building block, wherein the anti-codon corresponds to and identifies the polymer building block. The anti-codon hybridizes to at least one of the plurality of codons of the coding region of the linear initiator nucleic acid. Upon hybridization of the anti-codon, the polymer building block couples with the first initial building block or the second initial building block to form a covalent bond.

FIG. 8 shows a synthesized compound comprising a plurality of polymer building blocks extending from the first initial building block and a synthesized compound comprising a plurality of polymer building blocks extending from the second initial building block. The synthesized compound comprising the first initial building block and the synthesized compound comprising the second initial building block are the same as shown, but may be different.

### DETAILED DESCRIPTION OF THE INVENTION

In one aspect, the invention provides methods of making linear initiator nucleic acids. The linear initiator nucleic acids as described herein allow for synthesis of bivalent molecules (e.g., molecules which allow for the polydisplay of synthesized compounds), which increases the reactivity and target binding during downstream compound analyses. Pools of these bivalent molecules, each comprising synthesized compounds, may be screened for binding to targets. The target (e.g., a target protein) may be immobilized on a solid support and then incubated with the pool of bivalent molecules to allow for binding of certain bivalent molecules to the target. Those bivalent molecules which do not bind may then be washed away. Finally, those bivalent molecules bound to immobilized target may be identified, e.g., by sequencing of the oligonucleotide (which both identifies and encoded the synthesis of the synthesized compounds). Under conditions in which any two copies of the target protein are immobilized at a distance such that the two copies of a synthesized compound attached to the same bivalent molecule cannot both be bound at the same time, the synthesized compound of the bivalent molecule will have an apparent affinity for the target that is about twice the magnitude as a single copy of the synthesized compound for the target. When target proteins are immobilized close enough to each other such that both copies of a synthesized compound attached to the same bivalent molecule can simultaneously bind to two copies of the target, an avidity effect will cause the apparent affinity to be far greater in magnitude than that of a monovalent molecule comprising one copy of the synthesized compound. Thus, assays using bivalent molecules comprising at least two copies of the synthesized compound are more sensitive compared to assays using monovalent molecules, and reproducibly capture and help identify synthesized compounds with weaker affinities to the target protein. In screens involving thousands or even millions of candidate molecules, the use of these bivalent molecules helps to identify both strong binders for the target and moderate binders for the target. Candidates with only moderate binding can then be refined and optimized to increase their affinity for the target. The use of these bivalent molecules therefore allows for the identification of molecules which would otherwise be excluded from further development due to weak or moderate binding of the monovalent molecule to a target.

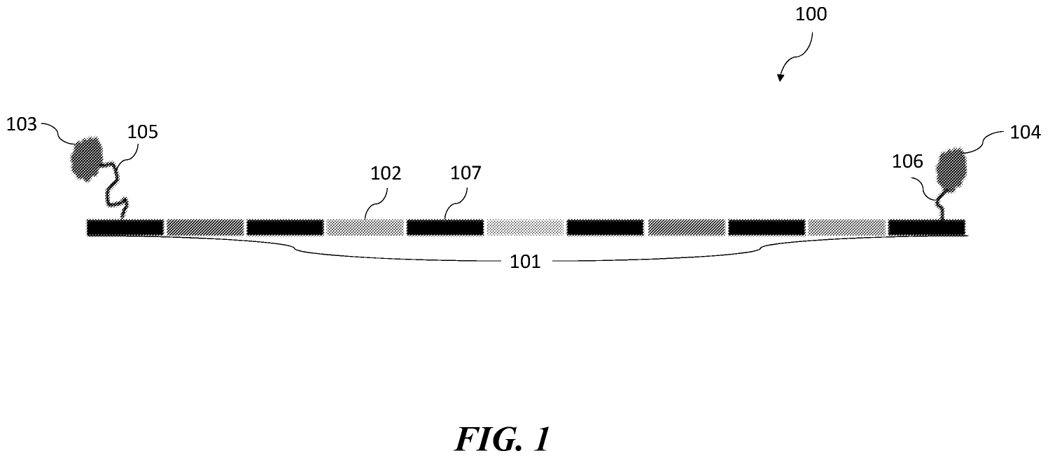

The bivalent molecules of the present invention are prepared from linear initiator nucleic acids. The linear initiator nucleic acid comprises a first building block, a second building block, and a coding region comprising a plurality of codons (see exemplary linear initiator nucleic acid at FIG. 1). The coding region corresponds to, and can be used to identify, the first initial building block and/or the second initial building block, in addition to polymer building blocks that attach to the initial building blocks after at least a first synthesis step. In some embodiments, the types of molecule or compound that can be used as an initial building block are not generally limited, so long as one initial building block is capable of reacting together with another polymer building block to form a covalent bond. In some embodiments, the first initial building block is the same as the second initial building block. In some embodiments, the first initial building block is not a nucleotide or derivative or polymer thereof. In some embodiments, the second initial building block is not a nucleotide or derivative or polymer thereof.

FIG. 1 shows an exemplary linear initiator nucleic acid 100 comprising a coding region 101 comprising a plurality of codons (such as 102). The linear initiator nucleic acid 100 may comprise additional non-coding regions (such as 107). The linear initiator nucleic acid 100 comprises an “upstream” first initial building block 103 which is connected by a linker 105 and a “downstream” second initial building block 104 which is connected by a linker 106. The first initial building block 103 may be, in some embodiments, the same chemical entity as the second initial building block 104. In some embodiments, the first initial building block 103 may be a different chemical entity than the second initial building block 104. The linker 105 and the linker 106 may be the same or different.

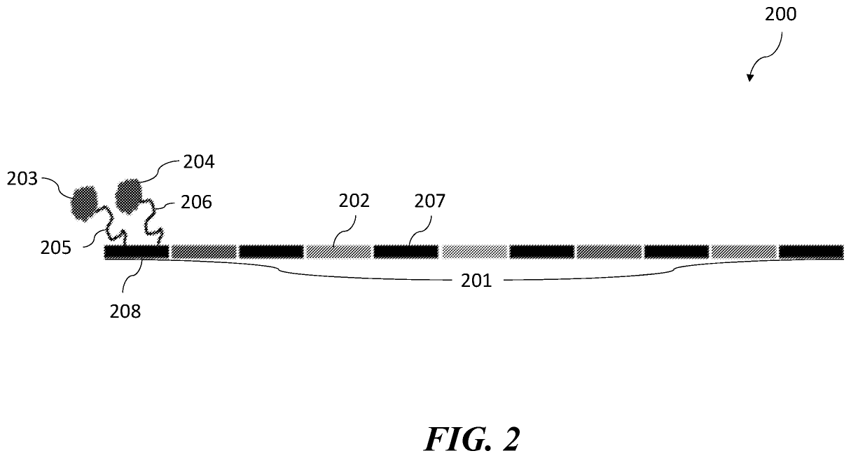

The linear initiator nucleic acids may be prepared from a linear precursor nucleic acid. FIG. 2 shows an exemplary linear precursor nucleic acid 200 which is useful for preparing the linear initiator nucleic acids described herein. The linear precursor nucleic acid 200 comprises a coding region 201 comprising a plurality of codons (such as 202) which is connected by a linker 206 to a first initial building block 204 and by a linker 205 to a second initial building block 203. The linear precursor nucleic acid 200 may comprise additional non-coding regions (such as 207). A cleavable linker 208 is positioned downstream of the second initial building block 203 and upstream of the first initial building block 204. The first initial building block 204 and the second initial building block 203 may be the same or different. The linker 206 and the linker 205 may be the same or different.

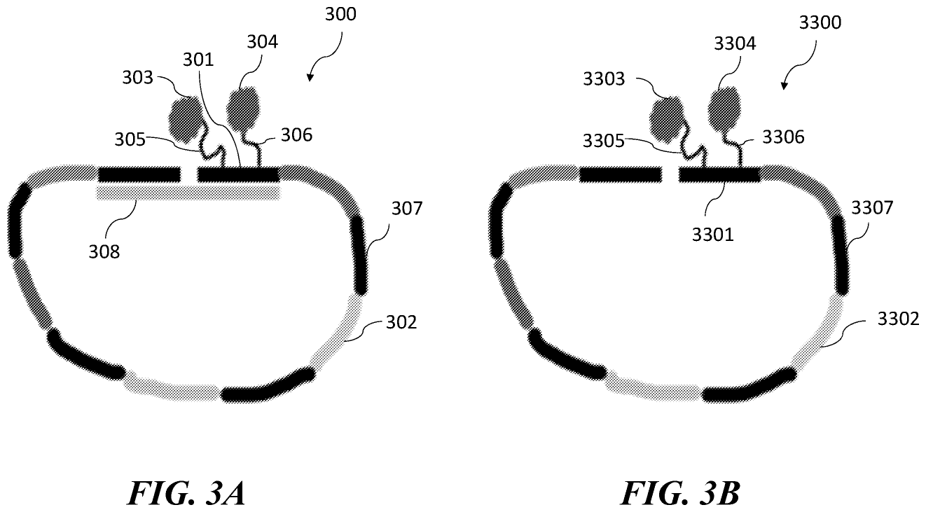

To form the linear initiator nucleic acids from a linear precursor nucleic acid, the linear precursor nucleic acid may form an intermediate non-covalently circularized nucleic acid. FIG. 3A shows an exemplary non-covalently circularized nucleic acid 300 comprising a coding region comprising a plurality of codons (such as 302). The non-covalently circularized nucleic 300 acid may comprise additional non-coding regions (such as 307). At a terminus of the nucleic acid a first initial building block 304 is connected by a linker 306 and a second initial building block 303 is connected by a linker 305. A cleavable linker 301 is positioned downstream of the second initial building block 303 and upstream of the first initial building block 304 (i.e., in between the two initial building blocks). The non-covalently circularized nucleic acid 300 is held in an orientation suitable for a ligation reaction of the termini by a splint 308. The splint 308 may be associated with the termini of the non-covalently circularized nucleic acid 300 by hybridization. The first initial building block 304 and the second initial building block 303 may be the same or different. The linker 306 and the linker 305 may be the same or different. FIG. 3B shows an additional exemplary non-covalently circularized nucleic acid 3300 comprising a coding region comprising a plurality of codons (such as 3302). The non-covalently circularized nucleic acid 3300 may comprise additional non-coding regions (such as 3307). The non-covalently circularized nucleic acid 3300 comprises a first initial building block 3304 connected by a linker 3306 and a second initial building block 3303 connected by a linker 3305. A cleavable linker 3301 is downstream of the second initial building block 3303 and upstream of the first initial building block 3304 (i.e., in between the two initial building blocks). The first initial building block 3304 and the second initial building block 3303 may be the same or different. The linker 3306 and the linker 3305 may be the same or different. The non-covalently circularized nucleic acid 3300 is illustrated in a spatial orientation suitable for blunt end ligation. It is understood that this orientation is transient, as is expected with blunt end ligation prior to the ligation step, and is not intended to show a stable orientation.

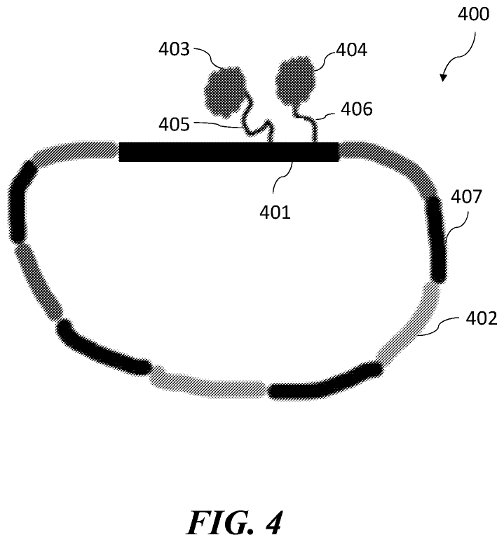

The non-covalently circularized nucleic acid may be covalently circularized by ligation to form a circularized nucleic acid. FIG. 4 shows an exemplary circularized nucleic acid 400 comprising a coding region comprising a plurality of codons (such as 402). The circularized nucleic acid 400 may comprise additional non-coding regions (such as 407). The circularized nucleic acid 400 comprises a first initial building block 404 connected by a linker 406 and a second initial building block 403 connected by a linker 405. A cleavable linker 401 is upstream of the first initial building block 404 and downstream of the second initial building block 403 (i.e., in between the two initial building blocks). The first initial building block 404 and the second initial building block 403 may be the same or different. The linker 406 and the linker 405 may be the same or different.

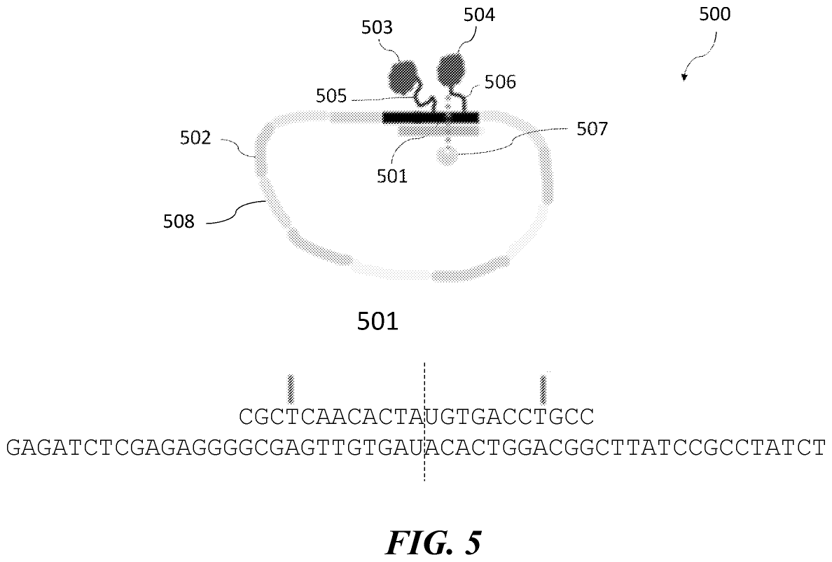

The linear initiator nucleic acids described herein are formed by cleavage of a cleavable linker in a circularized nucleic acid. FIG. 5 shows an exemplary circularized nucleic acid 500 comprising a coding region comprising a plurality of codons (such as 502). The linear initiator nucleic 500 may comprise additional non-coding regions (such as 508). The circularized nucleic acid comprises a first initial building block 504 connected by a linker 506 and a second initial building block 503 connected by a linker 505. A cleavable linker 501 is positioned upstream of the first initial building block 504 and the second initial building block 503. A reactive entity 507 (which may be an enzyme, such as a restriction enzyme, or a chemical capable of cleaving the cleavable linker) cleaves the cleavable linker 501 at a site (such as a restriction site; an exemplary restriction site is shown) to linearize the circularized nucleic acid 500. In this example, the circularized nucleic acid comprises a splint The splint hybridized to the circularized nucleic acid sequence forms the cleavable linker 501 (in this case, a restriction site) which may be cleaved (such as by a restriction site; an exemplary cut site is indicated by the dotted line in the sequences shown at the bottom of the figure). The sequence at the bottom of the figure shows a short primer containing amine-T (amine-T indicated by vertical bar over each T), which allow for attachment of the first and second initial building block. The bottom sequence is an exemplary splint. In a preliminary experiment, the exemplary splint and exemplary primer with amine-T attachment sites were efficiently cleaved by a restriction enzyme targeting the cute site indicated by the vertical dashed line (data not shown).

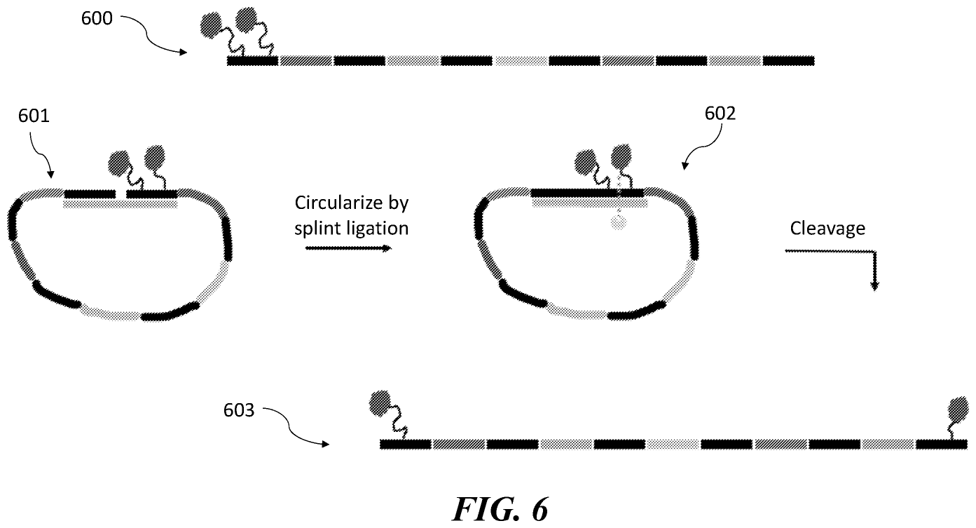

FIG. 6 shows an exemplary work flow for preparing a linear initiator nucleic acid 603 from a linear precursor nucleic acid 600. The linear precursor nucleic acid 600 forms a non-covalently circularized nucleic acid 601 (which, in this example, is by hybridization by a splint) and is covalently circularized by ligation (whether enzymatic or otherwise) to form a circularized nucleic acid 602. The circularized nucleic acid 602 is cleaved (for example, by activity of a restriction enzyme or by chemical cleavage) to form the linear initiator nucleic acid 603.

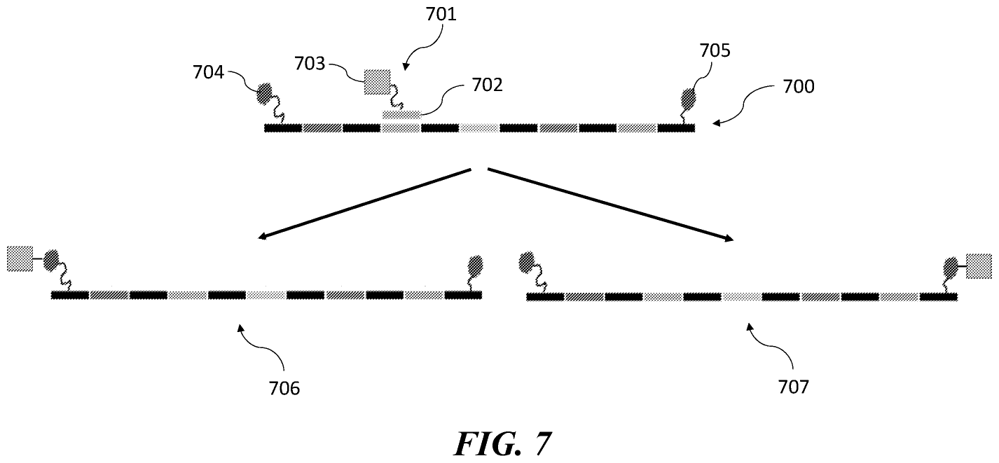

The linear initiator nucleic acids described herein are useful for preparing synthesized compounds. FIG. 7 shows an exemplary synthetic step (i.e., a step of adding a building block to the initial building blocks) in a method of synthesizing a compound (i.e., a bivalent molecule of the invention). A linear initiator nucleic acid 700 comprising a coding region comprising a plurality of codons is provided. The linear initiator nucleic acid 700 comprises a first initial building block 704 and a second initial building block 705. A charged anti-codon 701 comprising an anti-codon 702 and a polymer building block 703 hybridizes to a codon on the linear initiator nucleic acid 700. A coupling reaction occurs transferring the polymer building block to the first initial building block 705 or the second initial building block 704 to form a molecule 706 or 707. The process may be repeated to transfer a further polymer building block to either the first initial building block 705 or the second initial building block 704.

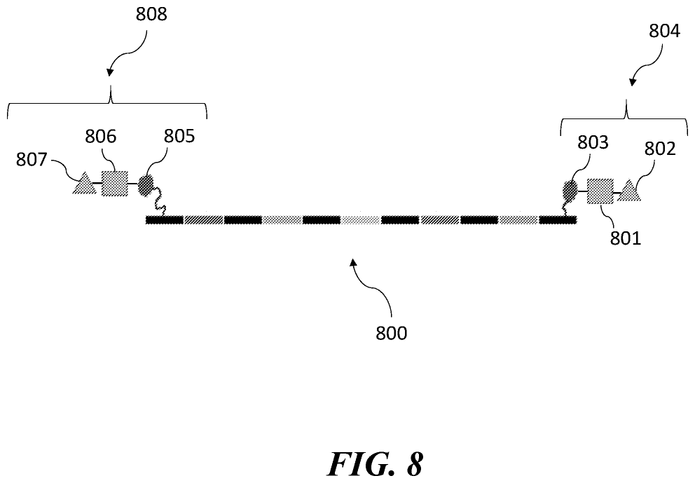

After a series of synthesis reactions, a synthesized compound may be formed from the linear initiator nucleic acids described herein. FIG. 8 shows an exemplary synthesized compound 800. The synthesized compound 800 comprises a first initial building block 803 and a second initial building block 805. The first initial building block 803 is coupled to a first polymer building block 801 and a second polymer building block 802; these three building blocks form a first encoded region 804. The second initial building block 805 is connected to a third polymer building block 806 and a fourth polymer building block 807; these three building blocks form a second encoded region 808. The first encoded region 804 and/or the second encoded region 808 may be assessed for desirable properties, such as ability to bind a target. The building blocks of the first encoded region 804 and the second encoded region 808 are identified by a coding region of the synthesized compound 800. The building blocks of the first encoded region 804 and the second encoded region 808 are shown as the same in FIG. 8, but may be different. The polymer building blocks 801, 802, 806, and 807 are exemplary and may be any suitable polymer building blocks. Therefore, a synthesized compound comprising the first encoded region 804 may be the same or different than a synthesized compound comprising the second encoded region 808.

In some embodiments, a first linker attaches the first initial building block to the linear initiator nucleic acid and a second linker attaches the second initial building block to the linear initiator nucleic acid. In some embodiments, the first linker and/or second linker is attached to the first initial building block and/or the second initial building block by a covalent bond. Various linkers are known in the art, and a first linker may be the same or different than a second linker. In some embodiments, the first initial building block is attached to a site that is upstream of the coding region on the initiator nucleic acid and the second initial building block is attached to a second site that is downstream of the coding region on the initiator nucleic acid. In some aspects, the building blocks are not nucleic acids or nucleic acid analogs.

The nucleic acids described herein comprise a coding region which comprises a plurality of codons. For example, the coding region may comprise from about 2 to about 20 codons, such as any of about 2 to about 10 codons, about 10 to 20 codons, about 5 to about 15 codons, about 10 to about 15 codons, and values and ranges therebetween. In some embodiments, the coding region comprises 2, 3, 4, 5, 6, 7, 8, 9, 10, 11, 12, 13, 14, 15, 16, 17, 18, 19, or 20 codons. In some embodiments, the coding region comprises from about 5 to about 20 codons. For example, a codon of a plurality of codons may comprise from about 8 to about 30 nucleotides. The codons may be used to encode (e.g., direct) the synthesis of a compound on a linear initiator nucleic acid, by encoding the addition of polymer building blocks. The polymer building blocks are added to one of the initial building blocks (e.g., the first initial building block and/or the second initial building block) or they are added to another polymer building block which are attached, directly or indirectly, to one of the initial building blocks. In either case, the codons of the coding region direct the addition of polymer building blocks to the linear initiator nucleic acid through a series of synthesis steps. The region comprising these polymer building blocks, including the initial building blocks, is termed an encoded region. In the case where the linear initiator nucleic acid has precisely two initial building blocks (i.e., a first initial building block and a second initial building block), then the molecule would have a first encoded region (comprising the first initial building block and one or more polymer building blocks) and a second encoded region (comprising the second initial building block and one or more polymer building blocks) after one or more synthesis steps directing the addition of one or more polymer buildings to each encoded region. In some embodiments, the polymer building blocks are not nucleic acids or nucleic acid analogs. In some embodiments, the types of molecules or compounds that can be used as a polymer building block are not generally limited, so long as one polymer building block is capable of reacting together with another polymer building block or initial building block to form a covalent bond.

In some embodiments, at least one codon encodes the addition of a polymer building block to the first initial building block, the second initial building block, or both. In some embodiments, the plurality of codons encodes for the addition of a plurality of polymer building blocks. Each polymer building blocks of a plurality of polymer building blocks may be different or may be the same. Alternatively, some polymer building blocks of a plurality of polymer building blocks may be the same, while other polymer building blocks of the plurality of polymer building blocks may be different.

The cleavable linker allows separation between the first initial building block and the second initial building block on a linear precursor nucleic acid and on the circularized nucleic acid (which is an intermediate molecule in the creation of the linear initiator nucleic acids described herein). The cleavable linker is oriented such that when a circularized nucleic acid is cleaved, whether by enzymatic cleavage (such as restriction digestion) or chemical cleavage, the first initial building block and the second initial building block are on (or near) opposite termini of the linear initiator nucleic acid. Thus, the cleavable linker may be a nucleic acid sequence (also termed an intervening sequence) between the sites of attachment of the first initial building block and the second initial building block, or it may be a chemically-cleavable linker between the sites of attachment of the first initial building block and the second initial building block.

In one aspect, a method of making the linear initiator nucleic acid comprises cleavage of a circularized nucleic acid, wherein the circular nucleic acids comprises the first initial building block, the second initial building block, a coding region, and a cleavable linker. An exemplary circular nucleic acid is provided in FIG. 4. In some embodiments, the cleavable linker comprises from about 2 to about 50 nucleotides. In some embodiments, the cleavable linker is a chemically-cleavable linker. In some embodiments, the cleavable linker is a chemically-cleavable linker joining two nucleotides in sequence. In some embodiments, the cleavable linker comprises a recognition sequence that is a restriction site, which may be cleaved by a restriction enzyme during restriction digestion. In some embodiments, the cleavable linker is an intervening sequence. In some embodiments, the intervening sequence is a nucleotide sequence. In some embodiments, the intervening sequence is from about 8 to about 30 nucleotides long. In some embodiments, the intervening sequence comprises a moiety that enable cleavage by an endonuclease. For example, the intervening sequence may comprise a base (e.g., a modified base, such as deoxyuridine (dU)) that enables cleavage by uracil DNA glycosylase (UDG) or formamidopyrimidine DNA glycosylase (FpG).

In the circularized nucleic acids described herein, the cleavable linker is positioned at a site between sites of the first initial building block and the second initial building block. As shown in FIG. 5, the cleavable linker of the circularized nucleic is cleaved. The cleavable linker is separated by cleavage, wherein a first portion of a cleavable linker (e.g., the 5′ end) and a second portion of a cleavable linker (e.g., the 3′ end) are separated. Cleavage of the cleavable linker may be by enzymatic cleavage (e.g, restriction digestion at a restriction site), or cleavage of the cleavable linker may be by chemical cleavage. Since the cleavable linker is positioned at a site that is between sites of the first initial building block and the second initial building block, the cleavage of the circularized nucleic acid occurs between the sites of the first initial building block and the second initial building block. Once the cleavable linker of the circularized nucleic acid is cleaved, a linear initiator nucleic acid is formed, wherein the first initial building block and the second initial building block are attached to opposite ends of the linear initiator nucleic acid. Therefore, the methods of making a linear initiator nucleic acid involve separating the first initial building block from the second initial building block such that they are on opposite ends of the linear initiator nucleic acid.

In another aspect, the invention provides a linear precursor nucleic acid. An exemplary linear precursor nucleic acid is illustrated in FIG. 2. The linear precursor nucleic acid may be used in a method of making a linear initiator nucleic acid. The method comprises circularizing the linear precursor nucleic acid, such as by ligation. The linear precursor nucleic acid comprises the first initial building block, the second initial building block, the coding region, and the cleavable linker. The site of the first initial building block and the site of the second initial building block are flanking the site of the cleavable linker on the linear precursor nucleic acid. In some embodiments, each of the first initial building block, the second initial building block, and the cleavable linker are downstream of the coding region of the linear precursor nucleic acid. In some embodiments, each of the first initial building block, the second initial building block, and the cleavable linker are upstream of the coding region of the linear precursor nucleic acid.

In some embodiments, the linear precursor nucleic acid (an exemplary linear precursor nucleic acid is illustrated in FIG. 2) is ligated to form the circularized nucleic acid (exemplary circularized nucleic acids are illustrated in FIG. 4; an exemplary method is illustrated in FIG. 6). In some embodiments, the ligation is splint ligation using a nucleotide splint. In some embodiments, the 5′ and 3′ termini of the precursor nucleic acid are non-covalently bound to a nucleotide splint. An exemplary non-covalently circularized nucleic acid comprising a splint is illustrated in FIG. 3A, e.g. 308. In some embodiments, the ligation is blunt end ligation that does not require the use of a nucleotide splint. An exemplary nucleic acid in an orientation suitable for blunt end ligation is illustrated in FIG. 3B. In some embodiments, the splint is removed following cleavage of the cleavable linker. Removal of the splint may be advantageous to downstream processes, because the splint is no longer required for synthesis of a linear initiator nucleic acid after cleavage of the cleavable linker occurs. In some embodiments, the removing of the splint comprises cleaving the splint. For example, the splint may be cleaved by incorporating one or more deoxyuridine (dU) bases into the splint, and subsequently digesting with uracil DNA glycosylase (UDG). In some embodiments, the cleavable linker and the splint both comprise one or more dU bases. In some embodiments, the dU base(s) of the cleavable linker and the splint are cleavage in the same reaction with UDG.

In another aspect, the invention provides a circularized nucleic acid. The circularized nucleic acid may be cleaved (e.g., the cleavable linker of the circularized nucleic acid may be cleaved) to form a linear initiator nucleic acid. The circularized nucleic acid comprises the first initial building block, the second initial building block, the cleavable linker, and the coding region. The cleavable linker is positioned at a site in between the sites of the first initial building block and the second initial building block on the circularized nucleic acid, such that cleavage of the cleavable linker results in sites of the first initial building block and the second initial building block being on opposite ends of the linear initiator nucleic acid.

In another aspect, the invention provides methods of synthesizing a compound. In some embodiments, synthesizing the compound results in a molecule that displays multiple copies of the compound (i.e., bivalent display or polyvalent display of the compound). For example, a linear initiator nucleic acid comprising a first initial building block at one end and a second initial building block at the opposite end is subjected to rounds of synthesis that add one or more polymer blocks to the first initial block and/or the second initial building block. The first initial building blocks and the attached polymer building blocks can be tested for desirable properties, such as binding to a target. Similarly, the second initial building block and the attached polymer building blocks can be tested for desirable properties, such as binding to a target. As used herein, reference to a “compound” can mean the first initial building block attached to one or more polymer building blocks and/or the second initial building block attached to one or more polymer building blocks.

The synthesis of the compound may be encoded (e.g., directed) by the coding region of the linear initiator nucleic acid. In some embodiments, the synthesized compound comprises the first initial building block. In some embodiments, the synthesized compound comprises the second initial building block. In some embodiments, the synthesized compound comprising the first initial building block is the same as the synthesized compound comprising the second initial building block.

The initiator nucleic acid comprising a first initial building block and a second initial building block, which are located at sites near opposite ends of the initiator nucleic acid, may be used to direct the synthesis of compounds at both the first initial building block and the second initial building block. Thus, a molecule is formed from the linear initiator nucleic acid which comprises two encoded regions. A first encoded region comprises a synthesized compound comprising the first initial building block and one or more polymer building blocks. A second encoded region comprises a synthesized compound comprising the second initial building block and one or more polymer building blocks. This system is intended to be flexible, and as such the first initial building block and the second initial building block may be the same or different. Further, the polymer building blocks attached to the first initial building block and the same initial building block may be the same or different.

In an exemplary embodiment, the first encoded region and the second encoded region comprise an identical chemical structure. For example, if the first initial building block and the second initial building block are the same, and the type and order of polymer building blocks attached to the first initial building block and second initial building block are the same (see, e.g., FIG. 8), then the overall molecule will have improved binding properties for certain target molecules. In an assay designed to identify compounds that bind a target, those compounds with weaker binding may be more efficiently identified when a molecule displays two or more copies of the same encoded region, as compared to a molecule displaying only a single copy of said encoded region.

In an additional exemplary embodiment, the first encoded region and the second encoded region comprise a different chemical structure. In some embodiments, the first initial building block of the first encoded region is different than the second initial building block of the second encoded region. In some embodiments, the type and/or order polymer building blocks attached to the first initial building block and the second initial building block are different. For example, if the first initial building block and the second initial building block are different, and the type and order of polymer building blocks attached to the first initial building block and second initial building block are different, then the total number of unique molecules in the DNA encoded library increases. Increasing the total number of unique molecules in the library similarly increases the likelihood that a molecule with desired properties will be detected (e.g., a target binding molecule). Additionally, a molecule comprising two distinct encoded regions doubles the number of synthesized compounds without increasing the number of nucleic acid strands in the system, which may be a limiting factor in the synthesis of DNA encoded libraries.

In some embodiments, a pool of molecules comprising a plurality of linear initiator nucleic acids is provided. An exemplary linear initiator nucleic acid of the pool of molecules is illustrated in FIG. 7 at 700. In some embodiments, at least one linear initiator nucleic acid of the plurality of linear initiator nucleic acids is made according to the methods provided by the present invention. In some embodiments, each linear initiator nucleic acid of the plurality of linear initiator nucleic acids is made according to the methods provided by the present invention. In some embodiments, the linear initiator nucleic acid is formed by cleavage of a circularized nucleic acid (e.g., by enzymatic cleavage or chemical cleavage of a cleavable linker). The circularized nucleic acid may be formed by ligation of the ends of a linear precursor nucleic acid. In some embodiments, the enzymatic cleavage comprises cleavage by an endonuclease. For example, the intervening sequence may comprise a base (e.g., a modified dU base) that enables cleavage by uracil DNA glycosylase (UDG) or formamidopyrimidine DNA glycosylase (FpG). In some embodiments, the enzymatic digestion comprises restriction digestion, and the restriction digestion occurs at a restriction site of an intervening sequence of the circularized nucleic acid. In some embodiments, the restriction digestion occurs at a restriction site of a cleavable linker of the circularized nucleic acid.

In some embodiments, each linear initiator nucleic acid of the plurality of linear initiator nucleic acids comprises a first initial building block, a second initial building block, and a coding region comprising a plurality of codons. The first initial building block may be attached to a site that is upstream of the coding region on the linear initiator nucleic acid and the second initial building block may be attached to a second site that is downstream of the coding region on the linear initiator nucleic acid.

In some embodiments of the method of synthesis of a compound as exemplified in FIGS. 7 and 8, at least one of the linear initiator nucleic acids is contacted with at least one charged anti-codon. A charged anti-codon is an anti-codon comprising a polymer building block. The anti-codon is capable of hybridizing with at least one of the codons of the coding region of the linear initiator nucleic acid. The anti-codon may not react with the non-coding regions. In some embodiments, the polymer building block of the anti-codon reacts with the first initial building block or the second initial building block of the linear initiator nucleic acid to form a covalent bond. In some embodiments, the reaction of a polymer building block with the first initial building block or the second initial building block produces a synthesized compound. In some embodiments, the anti-codon is removed (e.g., unhybridized) from the linear initiator nucleic acid following the reaction of the polymer building block with the first initial building block or the second initial building block. In some embodiments, the removal of the anti-codon is more efficient when the anti-codon comprises one or more modified bases (e.g., dU base(s)) that may be cleaved (e.g., by uracil DNA glycosylase (UDG)), thus cleaving and removing the anti-codon from the linear initiator nucleic acid. Removal of the anti-codon from the linear initiator nucleic acid allows for a second charged anti-codon comprising an anti-codon and a second copy of the polymer building block to hybridize to with at least one codon of the coding region of the linear initiator nucleic acid. Optionally, wherein the second charged anti-codon comprises an identical polymer building block to the first charged anti-codon, the second anti-codon may hybridize to the same codon of the coding region of the linear initiator nucleic acid as the first anti-codon. The second polymer building block of the second anti-codons reacts with the unreacted first initial building block or the second initial building block to form a covalent bond and produce a synthesized compound.

In some embodiments of the method of synthesis of a compound, one or more additional charged anti-codons comprising additional polymer building blocks hybridize to at least one of the codons of the coding region of the linear initiator nucleic acid, wherein the additional polymer building blocks react with the polymer building blocks extending from the first initial building block and/or the second initial building block. In some embodiments, a compound comprising a plurality of polymer building blocks, as exemplified in FIG. 8, extending from the first initial building block and a compound comprising a plurality of polymer building blocks extending from the second initial building block is synthesized by repeating the hybridization of anti-codons and reaction of polymer building blocks. In some embodiments, the synthesized compound extending from the first initial building block is the same as the synthesized compound extending from the second initial building block. In some embodiments, the synthesized compound extending from the first initial building block is different than the synthesized compound extending from the second initial building block. In some embodiments, the synthesized compound does not comprise a nucleic acid or nucleic acid analog.

The linear initiator nucleic acids provided by the methods herein may be used to prepare molecules comprising synthesized compounds, as shown in FIG. 8. The molecules are bifunctional or multifunctional and comprise the nucleic acid portion 800, which both encoded synthesis of the compounds (e.g., 808 and 804, which each the first and second encoded regions) and identifies the synthesized compounds, and further comprise the synthesized compounds (e.g., comprising initial and polymer building blocks). Importantly, the compounds may be identical or may be different, which confer different benefits as described above. The molecules, by virtue of having a plurality of synthesized compounds, i.e., bivalent or polyvalent display, improve the efficiency of screening of a library of synthesized compounds.

### Definitions

As used herein, the singular forms “a,” “an,” and “the” include the plural references unless the context clearly dictates otherwise.

Reference to “about” a value or parameter herein includes (and describes) variations that are directed to that value or parameter per se. For example, description referring to “about X” includes description of “X”.

It is understood that aspects and variations of the invention described herein include “consisting” and/or “consisting essentially of” aspects and variations.

Unless otherwise noted, the term “hybridize,” “hybridizing,” and “hybridized” includes Watson-Crick base pairing, which includes guanine-cytosine and adenine-thymine (G-C and A-T) pairing for DNA and guanine-cytosine and adenine-uracil (G-C and A-U) pairing for RNA. These terms are used in the context of the selective recognition of a strand of nucleotides for a complementary strand of nucleotides, called an anti-codon or anti-coding region which is complementary and hybridizes to a coding region.

The terms “end” and “terminus”, in the context of describing the position of a feature of the nucleic acids described herein, are used synonymously to mean a position that is near the absolute end or absolute terminus of a linear nucleic acid molecule. For example, an initial building block linked to any one of the 20 nucleic acids at the 5′ end of a nucleic acid may be described as being at a position at the “5′ end” or “5′ terminus” of the nucleic acid.

The term “bivalent molecule” refers to a multifunctional molecule that contains an oligonucleotide, at least one encoded portion, and at least two initial building blocks. A “polyvalent molecule” is used to describe a multifunctional molecule that contains an oligonucleotide, at least one encoded portion, and more than two building blocks (e.g., at least two initial building blocks and at least one polymer building blocks). In the context of a bivalent or polyvalent molecule, the at least two initial building blocks may be the same, and are not nucleic acids or nucleic acid analogs.

The “encoded portion” of a multifunctional molecule refers to a section of the “encoded region” of a multifunctional molecule. This encoded portion comprises polymer building blocks and comprises initial building blocks, whose attachment to the multifunctional molecule is encoded and/or directed by the codons of the coding region.

As used herein, the terms “upstream” and “downstream” are used to refer to relative positions of features on a sequence of DNA or RNA. “Upstream” is towards the 5′ end of the strand of DNA or RNA, and “downstream” is towards the 3′ end of the strand of DNA or RNA. When considering the positioning on a double-stranded DNA sequence, both “upstream” and “downstream” refer to the positioning on the coding strand of the oligonucleotide.

The term “coding region” is used to describe a region of the linear initiator nucleic acid that is used to identify the building blocks of the linear initiator nucleic acid. For example, the coding region may be an oligonucleotide that encodes and directs the synthesis of a compound, wherein the coding region determines which anti-codons comprising polymer building blocks may hybridize to the linear initiator nucleic acid, thereby synthesizing an encoded compound.

When a range of values is provided, it is to be understood that each intervening value between the upper and lower limit of that range, and any other stated or intervening value in that stated range, is encompassed within the scope of the present disclosure. Where the stated range includes upper or lower limits, ranges excluding either of those included limits are also included in the present disclosure.

The section headings used herein are for organization purposes only and are not to be construed as limiting the subject matter described. The description is presented to enable one of ordinary skill in the art to make and use the invention and is provided in the context of a patent application and its requirements. Various modifications to the described embodiments will be readily apparent to those persons skilled in the art and the generic principles herein may be applied to other embodiments. Thus, the present invention is not intended to be limited to the embodiment shown but is to be accorded the widest scope consistent with the principles and features described herein.

The disclosures of all publications, patents, and patent applications referred to herein are each hereby incorporated by reference in their entireties. To the extent that any reference incorporated by reference conflicts with the instant disclosure, the instant disclosure shall control.

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference. The section headings used herein are for organizational purposes only and are not to be construed as limiting the subject matter described.

### Methods of Making a Linear Initiator Nucleic Acid

The methods provided herein relate, in some aspects, to making linear initiator nucleic acids. An exemplary linear initiator nucleic acid is illustrated in FIG. 1. The linear initiator nucleic acid is an encoded molecule suitable for the polydisplay of building blocks (e.g., a synthesized compound). Polydisplay molecules are advantageous due to increased target binding compared to combinatorial libraries of compounds with a single copy of a building block. These linear initiator nucleic acid will not only increase reaction efficiency of the successive reaction steps for the creation of combinatorial libraries, but the resulting compounds (e.g., encoded molecules comprising synthesized compounds) will also be effective at binding targets even though the compounds and/or targets may be present in low numbers.

In some embodiments, the linear precursor nucleic acid comprises a first initial building block, a second initial building block and a coding region. A method of making a linear initiator nucleic acid as provided herein may begin with a linear precursor nucleic acid. The linear precursor nucleic acid comprises the first initial building block and the second initial building block attached to positions flanking a cleavable linker. Each of the first initial building block, the second initial building block, and the cleavable linker are all upstream or all downstream of the coding region in the linear precursor nucleic acid. The coding region, which comprises a plurality of codons, corresponds to, and can be used to identify, the first initiator building block and the second initiator building block, in addition to polymer building blocks that attach to the initiator building blocks.

The linear precursor nucleic acid may be circularized to form a circularized nucleic acid. In some embodiments, the linear precursor nucleic acid comprising a first initial building block and a second initial building block is circularized by splint ligation or blunt end ligation. In some embodiments, the circularization of the linear precursor nucleic acid is facilitated by splint ligation of the 3′ and 5′ termini of the linear precursor nucleic acid (as exemplified in FIG. 6). The resulting circularized nucleic acid comprises the first initial building block, the second initial building block, the cleavable linker, and the coding region. As illustrated in FIG. 6, the first initial building block and the second initial building block are attached to sites flanking the cleavable linker on the circularized nucleic acid.

The circularized nucleic acid may then be cleaved at the cleavable linker. In some embodiments, the circularized nucleic acid is cleaved at the cleavable linker by enzymatic cleavage or chemical cleavage. In some embodiments, the cleavable linker is positioned at a site in between the site of the first initial building block and the site of the second initial building block. In some embodiments, the cleavage occurs at the cleavable linker, wherein the cleavage occurs between the first initial building block and the second initial building block. In some embodiments, the cleavage of the circularized nucleic acid results in a linear initiator nucleic acid, wherein the first initial building block and the second initial building block are moved to sites near opposite ends of the nucleic acid. FIG. 6 illustrates that cleavage of the circularized nucleic acid occurs between the first initial building block and the second initial building block (e.g., at the cleavable linker), thus forming the linear initiator nucleic acid which comprises a first initial building block attached to a site that is upstream of the coding region and a second initial building block attached to a site that is downstream of the coding region.

A pool of linear initiator nucleic acids may then be used for the production of combinatorial chemical libraries, to encode and direct the synthesis of compounds extending from the first initial building block and from the second initial building block.

### Building Blocks

The linear initiator nucleic acids described herein are used to assemble synthesized compounds comprising building blocks. The linear initiator nucleic acid initially carries a first initial building block and a second initial building block. The coding region of the linear initiator nucleic acid then directs the addition of polymer building blocks to the first and second initial building blocks. This addition occurs by a series of synthesis steps, each adding these additional polymer building blocks in sequence. At a desired length, the initial building block (whether the first initial building block or the second initial building block) and its attached polymer building blocks form one of the encoded regions of the bivalent molecule. The encoded regions may then be screened for their ability to bind targets (such as a target protein). Those encoded regions which bind target protein may then be identified, for example, by sequencing the nucleic acid sequence which encoded the encoded regions. It is then possible to exchange one or more of the building blocks of a candidate encoded region by creating a new library of bivalent molecules and testing that library for more efficient binders to the target protein. Following these procedures allows for the identification of high affinity binders of particular targets.

A “building block” as used herein is a chemical structural unit capable of being chemically linked to other chemical structural units (e.g., other building blocks). A “building block” may mean an initial building block or a polymer building block. The methods of making a linear initiator nucleic acid described herein, in some aspects, require one or more building blocks. Building blocks may include initial building blocks or polymer building blocks. The polymer building block is attached to (i.e., coupled with) an initial building block. In some embodiments, the polymer building block is reacted with the initial building block to form a covalent bond. In some embodiments, the linear initiator nucleic acids described herein comprise one or more initial building blocks. In some embodiments, the linear initiator nucleic acid comprises a first initial building block and a second initial building block.

In some embodiments, the building blocks are not nucleic acids or nucleic acid analogs. In some embodiments, the initial building blocks are not nucleic acids or nucleic acid analogs. In some embodiments, the polymer building blocks are not nucleic acids or nucleic acid analogs. In some embodiments, a building block has one, two, or more reactive chemical groups that allow the building block to undergo a chemical reaction that links the building block to other chemical structural units (e.g., other chemical structural units present in other building block, such as polymer building blocks). In some embodiments, the building block is linked to other chemical structural units (e.g., other building blocks) by a covalent bond.

It is understood that part or all of the reactive chemical group of a building block may be lost when the building block undergoes a reaction to form a chemical linkage. For example, a building block in solution may have two reactive chemical groups. In this example, the building block in solution can be reacted with the reactive chemical group of a building block that is part of a chain of building blocks to increase the length of a chain, or extend a branch from the chain. When a building block is referred to in the context of a solution or as a reactant, then the building block will be understood to contain at least one reactive chemical group, but may contain two or more reactive chemical groups. When a building block is referred to the in the context of a polymer, oligomer, or molecule larger than the building block by itself, then the building block will be understood to have the structure of the building block as a (monomeric) unit of a larger molecule, even though one or more of the chemical reactive groups will have been reacted.

The types of molecule or compound that can be used as a building block are not generally limited, so long as one building block is capable of reacting together with another building block to form a covalent bond. In some embodiments, the building block is not a nucleic acid or nucleic acid analog. In some embodiments, the building block is a chemical structural unit.

In some embodiments, the building block has one chemical reactive group to serve as a terminal unit. In some embodiments, the building block has 1, 2, 3, 4, 5, or 6 suitable reactive chemical groups. In some embodiments, a first initiator building block, a second initiator building block, and a polymer building block each independently have 1, 2, 3, 4, 5, or 6 suitable reactive chemical groups. Suitable reactive chemical groups for building blocks include, a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a haloacetate, an aryl halide, an azide, a halide, a triflate, a diene, a dienophile, a boronic acid, an alkyne, and an alkene.

Any coupling chemistry can be used to connect building blocks (e.g., initial building blocks to polymer building blocks and polymer building blocks to polymer building blocks), provided that the coupling chemistry is compatible with the presence of an oligonucleotide.

Exemplary coupling chemistry includes, formation of amides by reaction of an amine, such as a DNA-linked amine, with an Fmoc-protected amino acid or other variously substituted carboxylic acids; formation of ureas by reaction of an amine, including a DNA-linked amine, with an isocyanate and another amine (ureation); formation of a carbamate by reaction of amine, including a DNA-linked amine, with a chloroformate (carbamoylation) and an alcohol; formation of a sulfonamide by reaction of an amine, including a DNA-linked amine, with a sulfonyl chloride; formation of a thiourea by reaction of an amine, including a DNA-linked amine, with thionocarbonate and another amine (thioureation); formation of an aniline by reaction of an amine, including a DNA-linked amine, with a heteroaryl halide (SNAr); formation of a secondary amine by reaction of an amine, including a DNA-linked amine, with an aldehyde followed by reduction (reductive animation); formation of a peptoid by acylation of an amine, including a DNA-linked amine, with chloroacetate followed by chloride displacement with another amine (an SN2 reaction); formation of an alkyne containing compound by acylation of an amine, including a DNA-linked amine, with a carboxylic acid substituted with an aryl halide, followed by displacement of the halide by a substituted alkyne (a Sonogashira reaction); formation of a biaryl compound by acylation of an amine, including a DNA-linked amine, with a carboxylic acid substituted with an aryl halide, followed by displacement of the halide by a substituted boronic acid (a Suzuki reaction); formation of a substituted triazine by reaction of an amine, including a DNA-linked amine, with a cyanuric chloride followed by reaction with another amine, a phenol, or a thiol (cyanurylation, Aromatic Substitution); formation of secondary amines by acylation of an amine including a DNA-linked amine, with a carboxylic acid substituted with a suitable leaving group like a halide or triflate, followed by displacement of the leaving group with another amine (SN2/SN1 reaction); and formation of cyclic compounds by substituting an amine with a compound bearing an alkene or alkyne and reacting the product with an azide, or alkene (Diels-Alder and Huisgen reactions). In certain embodiments of the reactions, the molecule reacting with the amine group, including a primary amine, a secondary amine, a carboxylic acid, a primary alcohol, an ester, a thiol, an isocyanate, a chloroformate, a sulfonyl chloride, a thionocarbonate, a heteroaryl halide, an aldehyde, a chloroacetate, an aryl halide, an alkene, halides, a boronic acid, an alkyne, and an alkene, has a molecular weight of from about 30 to about 330 Daltons.

In some embodiments of the coupling reaction, the building block might be added by substituting an amine, including a DNA-linked amine, using any of the chemistries above with molecules bearing secondary reactive groups like amines, thiols, halides, boronic acids, alkynes, or alkenes. Then the secondary reactive groups can be reacted with building blocks bearing appropriate reactive groups. Exemplary secondary reactive group coupling chemistries include, acylation of the amine, including a DNA-linked amine, with an Fmoc-amino acid followed by removal of the protecting group and reductive animation of the newly deprotected amine with an aldehyde and a borohydride; reductive animation of the amine, including a DNA-linked amine, with an aldehyde and a borohydride followed by reaction of the now-substituted amine with cyanuric chloride, followed by displacement of another chloride from triazine with a thiol, phenol, or another amine; acylation of the amine, including a DNA-linked amine, with a carboxylic acid substituted by a heteroaryl halide followed by an SNAr reaction with another amine or thiol to displace the halide and form an aniline or thioether; and acylation of the amine, including a DNA-linked amine, with a carboxylic acid substituted by a haloaromatic group followed by substitution of the halide by an alkyne in a Sonogashira reaction; or substitution of the halide by an aryl group in a boronic ester-mediated Suzuki reaction.

In some embodiments, the coupling chemistries are based on suitable bond-forming reactions known in the art. See, for example, March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al, Principles and Applications of Organotransition Metal Chemistry, University Science Books, Mill Valley, Calif. (1987), Chapters 13 to 20; each of which is incorporated herein by reference in its entirety.

In some embodiments, the building block can include one or more functional groups in addition to the reactive group or groups employed to attach (e.g., react) a building block. One or more of these additional functional groups can be protected to prevent undesired reactions of these functional groups. Suitable protecting groups are known in the art for a variety of functional groups (Greene and Wuts, Protective Groups in Organic Synthesis, second edition, New York: John Wiley and Sons (1991), incorporated herein by reference in its entirety). Particularly useful protecting groups include t-butyl esters and ethers, acetals, trityl ethers and amines, acetyl esters, trimethylsilyl ethers, trichloroethyl ethers and esters and carbamates.

The type of building block is not generally limited, so long as the building block is compatible with one more reactive groups capable of forming a covalent bond with other building blocks. In some embodiments, the building block is not a nucleic acid or nucleic acid analog.

Suitable building blocks include but are not limited to, a peptide, a saccharide, a glycolipid, a lipid, a proteoglycan, a glycopeptide, a sulfonamide, a nucleoprotein, a urea, a carbamate, a vinylogous polypeptide, an amide, a vinylogous sulfonamide peptide, an ester, a saccharide, a carbonate, a peptidylphosphonate, an azatides, a peptoid (oligo N-substituted glycine), an ether, an ethoxyformacetal oligomer, thioether, an ethylene, an ethylene glycol, disulfide, an arylene sulfide, a nucleotide, a morpholino, an imine, a pyrrolinone, an ethyleneimine, an acetate, a styrene, an acetylene, a vinyl, a phospholipid, a siloxane, an isocyanide, a isocyanate, and a methacrylate. In certain embodiments, the (BI)M or (B2)κ of formula (I) each independently represents a polymer of these building blocks having M or K units, respectively, including a polypeptide, a polysaccharide, a polyglycolipid, a polylipid, a polyproteoglycan, a polyglycopeptide, a polysulfonamide, a polynucleoprotein, a polyurea, a poly carbamate, a polyvinylogous polypeptide, a polyamide, a poly vinylogous sulfonamide peptide, a polyester, a polysaccharide, a polycarbonate, a polypeptidylphosphonate, a polyazatides, a polypeptoid (oligo N-substituted glycine), a polyethers, a polythoxyformacetal oligomer, a polythioether, a polyethylene, a polyethylene glycol, a poly disulfide, a polyarylene sulfide, a polynucleotide, a polymorpholino, a polyimine, a polypyrrolinone, a polyethyleneimine, a polyacetates, a polystyrene, a polyacetylene, a polyvinyl, a polyphospholipids, a polysiloxane, a polyisocyanide, a polyisocyanate, and a polymethacrylate. In certain embodiments of the molecule for formula (I), from about 50 to about 100, including from about 60 to about 95, and including from about 70 to about 90% of the building blocks have a molecular weight of from about 30 to about 500 Daltons, including from about 40 to about 350 Daltons, including from about 50 to about 200 Daltons.

It is understood that building blocks having two reactive groups would form a linear oligomeric or polymeric structure, or a linear non-polymeric molecule, containing each building block as a unit. It is also understood that building blocks having three or more reactive groups could form molecules with branches at each building block having three or more reactive groups.

A building block as described herein may be attached to a linear initiator nucleic acid, or precursor molecules thereof (e.g., a linear precursor nucleic acid or a circularized nucleic acid). In some embodiments, one or more initial building blocks are attached to the linear initiator nucleic acid. In some embodiments, one or more initial building blocks are attached to the linear initiator nucleic acid at a specific site relative to the coding region on the linear initiator nucleic acid. In some embodiments, the first initial building block is attached to a first site that is upstream of the coding region on the linear initiator nucleic acid. In some embodiments, the second initial building block is attached to a second site that is downstream of the coding region on the initiator nucleic acid. Alternatively, the first initial building block may be attached to a first site that is downstream of the coding region on the linear initiator nucleic acid, and the second initial building block is attached to a second site that is upstream of the coding region on the linear initiator nucleic acid.

In some embodiments of the methods described herein, the building block is attached to a linear initiator nucleic acid, a circularized nucleic acid, and/or a linear precursor nucleic acid as described below. In some embodiments, the building block is attached to a linear initiator nucleic acid, or precursors thereof, by a linker. In some embodiments, the linear initiator nucleic acid, or precursors thereof, comprises a first linker and a second linker. In some embodiments, the linear initiator nucleic acid, or precursors thereof, comprises two or more linkers. The term “linker” as used herein refers to a bifunctional molecule or a portion thereof, which attaches a building block to the linear initiator nucleic acid, or precursors thereof.

In some embodiments, the first linker attaches the first initial building block to the linear initiator nucleic acid, or precursors thereof. In some embodiments, the second linker attaches the second initial building block to the linear initiator nucleic acid, or precursors thereof. In some embodiments, the building block is attached to the linker (e.g., a first linker or a second linker) by a covalent bond. In some embodiments, the first initial building block is attached to the first linker by a covalent bond. In some embodiments, the second initial building block is attached to the second linker by a covalent bond. In some embodiments, the first linker is the same as second linker. In some embodiments, the first linker is different from the second linker.

Various commercially available linkers are amenable to the applications of the present methods. Example of linkers may include, but are not limited to, PEG (e.g., azido-PEG-NHS, or azido-PEG-amine, or di-azido-PEG), or an alkane acid chain moiety (e.g., 5-azidopentanoic acid, (S)-2-(azidomethyl)-1-Boc-pyrrolidine, 4-azidoaniline, or 4-azido-butan-l-oic acid N-hydroxysuccinimide ester); thiol-reactive linkers, such as those being PEG (e.g., SM(PEG)n NHS-PEG-maleimide), alkane chains (e.g., 3-(pyridin-2-yldisulfanyl)-propionic acid-Osu or sulfosuccinimidyl 6-(3′-[2-pyridyldithio]-propionamido)hexanoate)); and amidites for oligonucleotide synthesis, such as amino modifiers (e.g., 6-(trifluoroacetylamino)-hexyl-(2-cyanoethyl)-(N,N diisopropyl)-phosphoramidite), thiol modifiers (e.g., 5-trityl-6-mercaptohexyl-l-[(2-cyanoethyl)-(N,N-diisopropyl)]-phosphoramidite, or chemically co-reactive pair modifiers (e.g., 6-hexyn-1-yl-(2-cyanoethyl)-(N,N-diisopropyl)-phosphoramidite, 3-dimethoxytrityloxy-2-(3-(3-propargyloxypropanamido)propanamido)propyl-l-0-succinoyl, long chain alkylamino CPG, or 4-azido-butan-l-oic acid N-hydroxysuccinimide ester)); and compatible combinations thereof.

A building block may comprise an initial building block or a polymer building block. In some embodiments, the polymer building block is attached to an initial building block. In some embodiments, at least one polymer building block is attached to an initial block. In some embodiments, at least one polymer building block is attached to the first initial block. In some embodiments, at least one polymer building block is attached to the second initial block. In some embodiments, a plurality of polymer building blocks are attached to (e.g., extend from) an initial building block. In some embodiments, a plurality of polymer building blocks are attached to (e.g., extend from) a first building block. In some embodiments, a plurality of polymer building blocks are attached to (e.g., extend from) a second building block. In some embodiments, the attaching of a polymer building block to an initial building block comprises reacting the polymer building block with an initial building block. In some embodiments, the reacting comprises the formation of a covalent bond.

Many kinds of chemistry are available for use in this invention (e.g., for reaction of an initial building block with a polymer building block and for reaction of a polymer building block with another polymer building block). In theory, any chemical reaction could be used that does not chemically alter DNA. Reactions that are known to be DNA compatible include but are not limited to: Wittig reactions, Heck reactions, homer-Wads-worth-Emmons reactions, Henry reactions, Suzuki couplings, Sonogashira couplings, Huisgen reactions, reductive aminations, reductive alkylations, peptide bond reactions, peptoid bond forming reactions, acylations, SN2 reactions, SNAr reactions, sulfonylations, ureations, thioureations, carbamoylations, formation of benzimidazoles, imidazolidinones, quinazolinones, isoindolinones, thiazoles, imidazopyridines, diol cleavages to form glyoxals, Diels-Alder reactions, indole-styrene couplings, Michael additions, alkene-alkyne oxidative couplings, aldol reactions, Fmoc-deprotections, trifluoroacetamide deprotections, Alloc-deprotections, Nvoc deprotections and Boc-deprotections. (See, Handbook for DNA-Encoded Chemistry (Goodnow R. A., Jr., Ed.) pp 319-347, 2014 Wiley, N.Y. March, Advanced Organic Chemistry, fourth edition, New York: John Wiley and Sons (1992), Chapters 10 to 16; Carey and Sundberg, Advanced Organic Chemistry, Part B, Plenum (1990), Chapters 1-11; and Coltman et al., Principles and Applications of Organotransition Metal Chemistry, University Science Books, Mill Valley, Calif (1987), Chapters 13 to 20; each of which is incorporated herein by reference in its entirety.)

### Circularized Nucleic Acid and Linear Precursor Nucleic Acid

Further described herein are circularized nucleic acids. The circularized nucleic acids may be formed from a linear precursor nucleic acid described herein. The linear precursor nucleic acid is circularized by ligation to form the circularized nucleic acid. Cleavage of the circularized nucleic acid results in a linear initiator nucleic acid. In some embodiments, provided herein is a method of making a linear initiator nucleic acid comprising cleavage of the circularized nucleic acid to form the initiator nucleic acid. The circularized nucleic acid comprises a first initial building block, a cleavable linker, a second initial building block, and a coding region. An exemplary circularized nucleic acid is illustrated in FIG. 4. The first initial building block and the second initial building block flank a cleavable linker of the circularized nucleic acid.

The circularized nucleic acid may be formed from a linear precursor nucleic acid. The linear precursor nucleic acid comprises a first initial building block, a cleavable linker, a second initial building block, and a coding region. An exemplary linear precursor nucleic acid is illustrated in FIG. 2. The first initial building block and the second initial building block are attached at sites flanking the cleavable linker on the linear precursor nucleic acid. In some embodiments, each of the first initial building block, the second initial building block, and the cleavable linker are upstream of the coding region on the linear precursor nucleic acid. In some embodiments, each of the first initial building block, the second initial building block, and the cleavable linker are downstream of the coding region on the linear precursor nucleic acid. In some embodiments, the circularized nucleic acid is formed by circularizing (such as by ligation) a linear precursor nucleic acid.

In some embodiments, the linear precursor nucleic acid may be formed from a strand of RNA (which may be generated from a dsDNA template) comprising sequence corresponding to (such as complementary to) the coding region of the linear precursor nucleic acid. In an exemplary method of forming the linear precursor nucleic acid, a nucleic acid primer comprising two modified bases may be obtained, and two initial building blocks (e.g., a first initial building block and a second initial building block) may each be coupled to a modified base. The primer comprising the initial building blocks is then used as a primer for a reverse transcription reaction with the strand of RNA comprising sequence corresponding to the coding region of the linear precursor nucleic acid, thereby forming a RNA/DNA heteroduplex. In some embodiments, the heteroduplex of RNA/DNA is cleaved (e.g., by heat, by heat and base, or with appropriate RNases such as, but not limited to, RNase I, RNase A, or RNase H), leaving behind a single stranded DNA, thus forming the linear precursor molecule comprising the first initial building block, the second initial building block, the cleavable linker or the intervening sequence comprising the cleavable linker, and the coding region. In another exemplary method of forming the linear precursor nucleic acid, an oligonucleotide comprising the two modified bases may be obtained and may then be ligated with additional single-stranded DNA oligonucleotides (which may be synthesized or purchased) to form the linear precursor nucleic acid. In yet another exemplary method of forming the linear precursor nucleic acid, asymmetric PCR may be used to preferentially amplify the coding strand of a dsDNA template. The product of the asymmetric PCR may then be ligated with an oligonucleotide comprising the modified bases (which are the attachment sites for the first initial building block and the second initial building block). The dsDNA template may be prepared using any suitable means in the art.

The linear precursor molecule may be ligated to form a circularized nucleic acid. In some embodiments, the 5′ and 3′ termini of the linear precursor molecule are ligated to form the circular nucleic acid. In some embodiments, a ligase is used to ligate the 3′ and 5′ ends of the linear precursor nucleic acid.

In some embodiments, the ligation is a ligation through enzymatic means (e.g., a ligase to perform an enzymatic ligation). In some embodiments, the ligation involves chemical ligation. In some embodiments, the ligation involves template dependent ligation. In some embodiments, the ligation involves template independent ligation.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together (e.g., the termini of the linear precursor nucleic acid). Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies), and phage ligases such as T3 DNA ligase, T4 DNA ligase, and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has a DNA-splinted DNA ligase activity. In some embodiments, the ligase is a ligase that has an RNA-splinted DNA ligase activity.

In some embodiments, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation is blunt ligation. An exemplary linear precursor nucleic acid in an orientation suitable for blunt end ligation is shown in FIG. 3B. Blunt ligation involves the ligation of nucleic acid molecules lacking a single-stranded overhang at the site of ligation.

In some embodiments, the ligation of the linear precursor nucleic acid is a splint ligation, wherein the ligation is carried out using a ligase and a splint complementary to the nucleic acid molecules of the linear precursor nucleic acid. As exemplified in FIG. 3A, in some embodiments, the linear precursor nucleic acid hybridizes with a splint. In some embodiments, the splint is a nucleotide splint. In some embodiments, the linear precursor nucleic acid is bound to the nucleotide splint. In some embodiments, the linear precursor nucleic acid is non-covalently bound to the nucleotide splint. In some embodiments, the 5′ and 3′ termini of the linear precursor nucleic acid are non-covalently bound to the nucleotide splint.

In some embodiments, the splint hybridizes to, or near, the 5′ and 3′ termini of the linear precursor nucleic acid. In some embodiments, the annealed region at or near the 5′ and 3′ termini of the linear precursor nucleic acid have different properties. For example, the annealed region at or near the 5′ and 3′ termini of the linear precursor nucleic acid may have different melting temperatures (Tₘ), different sequences, different lengths, etc. In some embodiments, the splint may have a higher Tₘ with one annealed region (e.g., at or near the 5′ and 3′ termini) of the linear precursor nucleic acid compared to the other end of the linear precursor nucleic acid. In some embodiments, the Tₘ of the splint is chosen to promote intramolecular annealing between the splint and the linear precursor nucleic acid, in contrast to intermolecular annealing between splint oligonucleotides. In some embodiments, the intramolecular annealing/cyclization ligation of the linear precursor nucleic acid is 10 to 100 times more likely to occur than the intermolecular annealing/cyclization ligation of the splint itself. In some embodiments, the splint may hybridize to a greater number of nucleic acids on one end (e.g., the 5′ and 3′ termini) of the linear precursor nucleic acid compared to the other end of the linear precursor nucleic acid. Thus, in some embodiments, the splint hybridizes at or near to the 5′ and 3′ termini of the linear precursor nucleic acid asymmetrically (e.g., with different Tₘ and/or with a different length of splint nucleotides hybridizing to the linear precursor nucleic acid). For example, one end of the splint may have a Tₘ that is higher than the temperature at which the ligation of the linear precursor nucleic acid is conducted, and the other end of the splint may have a Tₘ that is similar to the temperature at which the ligation of the linear precursor nucleic acid is conducted. In some embodiments, the difference in Tₘ and/or sequence hybridization length between the ends of the splint-linear precursor nucleic acid hybridization complex, promotes effective cyclization ligation of the linear precursor nucleic acid, as described above. In some embodiments, the splint anneals near the 5′ and 3′ termini, such that there is a single-stranded non-complementary region comprised of the two ends of the linear precursor nucleic acid connected by the splint. In some embodiments, these non-templated ends can be ligated by enzymes capable of ligating single-stranded DNA such as CIRCLIGASE™ (Lucigen, Wisconsin, USA) or Thermostable 5′ App DNA/RNA ligase (New England BioLabs, Massachusetts, USA). In some embodiments, no splint is used, and cyclization is directly effected using a ligase such as the Thermostable 5′ App DNA/RNA ligase. One of skill in the art will recognize the need to first adenylate the 5′ end of the linear precursor nucleic acid. Further, one of skill in the art will recognize the possibility of intermolecular ligation when a splint is not used. Means of purifying the intramolecular ligation product from the intermolecular ligation product are known in the art. In some embodiments, ligation is effected with T4 RNA ligase (it will be appreciated by one skilled in the art that use of this enzyme will require a splint comprised of RNA). Other enzymatic methods exist for ligating the 5′ end and the 3′ end of the linear precursor to each other, and any suitable means of operably linking these ends may be selected.

In some embodiments, the nucleotide splint is between about 8 and about 100 nucleotides in length. In some embodiments, the linear precursor nucleic acid is hybridized to between about 10 and about 100 nucleotides of the splint. In some embodiments, the splint is hybridized to the linear precursor nucleic acid from about 4 to about 40 nucleotides on one side of the ligation site, and from about 4 to about 40 nucleotide on the other side of the ligation site. In some embodiments, the splint is bound to the linear precursor nucleic acid with the same number of nucleotides on both sides of the ligation site. In some embodiments, the splint is bound to the linear precursor nucleic acid with a different number of nucleotides on both sides of the ligation site (e.g., asymmetrically hybridized). In some embodiments, asymmetric hybridization of the splint to the linear precursor nucleic acid is advantageous to the circularization ligation of the linear precursor nucleic acid, as described above.

### Coding Region and Optional Non-Coding Region

The nucleic acids described herein comprise a coding region comprising a plurality of codons, and optionally non-coding regions. The coding region may be used to accurately identify the building blocks (e.g., the initiator building blocks and/or the polymer building blocks) of the linear initiator molecule, and compounds synthesized therefrom, during downstream analysis. In some embodiments, the coding region includes or is an oligonucleotide. The coding region may encode and direct the synthesis of a compound from a linear initiator nucleic acid; the coding region determines which anti-codons comprising polymer building blocks may hybridize to the linear initiator nucleic acid, and therefore which polymer building block react with initial building blocks and/or polymer building extending from initial building blocks, to synthesize a specifically encoded compound. Additional description of coding region(s) and optional non-coding region(s) can be found in US 2020/0263163 A1 and US 2019/0169607 A1, which are hereby incorporated by reference in their entirety for all purposes.

In some embodiments, the coding region contains from about 1% to 100%, such as any of about 50% to about 100% or about 90% to about 100%, single stranded oligonucleotide.

The linear initiator nucleic acid comprising a first initial building block and a second initial building block may be used to synthesize compounds. The compounds are formed by attaching polymer building blocks to the first initial building block and the second initial building block. The polymer building blocks are added by first attaching them each to an anti-codon, to form a charged anti-codon, and then hybridizing the charged anti-codon to a codon in the coding region of the linear initiator nucleic acid. The polymer building block is then transferred from the anti-codon to the encoded region of the linear initiator nucleic acid (either by coupling to an initial building block or by coupling to a polymer building block). Generally any polymer building block can be attached to any anti-codon. Thus, if the sequence of the linear initiator nucleic acid is known (which can be determined by PCR), and the polymer building blocks used for each unique anti-codon during synthesis of the compound are known, then the identity of the synthesized compound can be determined.

In some embodiments, the linear initiator nucleic acid comprises at least one coding region comprising at least two codons, wherein the at least two codons correspond to and can be used to identify a building block in the linear initiator nucleic acid or compounds synthesized therefrom. In some embodiments, the at least one coding region can be amplified by PCR to produce copies of the at least one coding region and the original or copies can be sequenced to determine the sequence of the at least one coding region of the linear initiator nucleic acid. The determined sequence can be used to identify the identity of the initial building blocks and the polymer building blocks extending therefrom. In some embodiments, the sequence of the coding regions can be correlated to the series of combinatorial chemistry steps used to synthesize the synthesized compound (such as the initial building blocks and polymer building blocks extending therefrom).

In some embodiments, the coding region is double stranded. In some embodiments, the coding region is single stranded. The coding region comprises a plurality of codons. The number of codons in the coding region determines how many unique anti-codons the coding region can hybridize with. If the number of codons is below 2, the encoded portion may be too small to be practical. If the number of codons is too far above 20, synthetic inefficiencies may interfere with accurate synthesis. Thus, the number of codons is typically a value between these lower and upper bounds. In some embodiments, the coding region comprises between about 2 to about 21 codons, such as between any of about 2 to about 20 codons, about 5 to about 15 codons, and about 10 to about 21 codons. In some embodiments, the coding region comprises less than about 21 codons, such as less than about any of about 20, 15, 5, or 3 codons. In some embodiments, the coding region comprises about 2, 3, 4, 5, 6, 7, 8, 9, 10, 11, 12, 13, 14, 15, 16, 17, 18, 19, 20, or 21 codons. In some embodiments, the coding region comprises between about 5 to about 20 codons. In some embodiments, the codons of the coding regions may overlap with one another.

DNA-encoded synthesis uses the above-described codons to hybridize with anti-codons. The codons used in DNA-encoded synthesis are typically longer than those used in nature (i.e., those which are scanned by a ribosome along an mRNA). If a codon is less than about 6 nucleotides in length, the codon may not accurately direct synthesis of the encoded region. If a codon is too long, such as more than about 50 nucleotides, the codon may become cross-reactive. Such cross reactivity would interfere with the ability of the coding regions to accurately direct and identify the synthesis steps used to synthesize the coding region of the linear nucleic acid. Thus, in some embodiments, each codon of the plurality of codons of a coding region comprises between about 6 to about 50 nucleotides, such as between any of about 6 to about 20, about 8 to about 30, about 15 to about 25, and about 30 to about 50 nucleotides. In some embodiments, each codon comprises less than about 50 nucleotides, such as less than any of about 45, 40, 35, 30, 25, 20, 15, 10, or 6 nucleotides. In some embodiments, each codon comprises about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides. In some embodiments, each codon comprises between about 8 and about 30 nucleotides.

In some embodiments, the one or more codons of the coding region overlap. In some embodiments, at least two of the codons of the coding region overlap so as to be coextensive, provided that the overlapping codons only share from about 30% to 1% of the same nucleotides, including about 20% to 1%, including from about 10% to 2%. In some embodiments of the linear initiator nucleic acid, the coding region is from about 30% to 100%, including about from 60% to 100%, including about from 80% to 100%, single stranded. In some embodiments, the linear initiator nucleic acid comprising at least two coding regions comprising at least one codon each, wherein at least two of the coding regions are adjacent. In some embodiments, the linear initiator nucleic acid comprises at least two coding regions, wherein the at least two coding regions are separated by regions of nucleotides that do not direct or record synthesis of an encoded portion of the linear initiator nucleic acid (e.g., a synthesized compound).

The linear initiator nucleic acid may direct the synthesis of a compound by selectively hybridizing to a complementary anti-codon comprising a polymer building block (i.e., a charged anti-codon). In some embodiments, a codon of the coding region is unique to (e.g., corresponds to) the identity of a polymer building block that is attached to an initial building block. In some embodiments, the anti-codon comprises a polymer building block and at least one corresponding anti-codon which hybridizes with at least one of the plurality of codons in the coding region.

In some embodiments, at least one codon in the coding region of the linear initiator nucleic acid encodes the addition of a polymer building block to an initial building block. In some embodiments, at least one codon encodes the addition of a polymer building block to the first initial building block. In some embodiments, at least one codon encodes the addition of a polymer building block to the second initial building block. In some embodiments, at least one codon encodes the addition of a polymer building block to the first initial building block and the second initial building block. In some embodiments, at least one codon encodes the addition of a polymer building block to the first initial building block or the second initial building block.

In some embodiments, at least one codon of a plurality of codons encodes for the addition of one polymer building block of a plurality of polymer building blocks. In some embodiments, each codon of a plurality of codons encodes for the addition of one polymer building block of a plurality of polymer building blocks. In some embodiments, a plurality of codons encodes for the addition of a plurality of polymer building blocks.

In some embodiments, the coding region can contain natural and unnatural nucleotides. Suitable nucleotides include the natural nucleotides of DNA (deoxyribonucleic acid), including adenine (A), guanine (G), cytosine (C), and thymine (T), and the natural nucleotides of RNA (ribonucleic acid), adenine (A), uracil (U), guanine (G), and cytosine (C). Other suitable bases include natural bases, such as deoxyadenosine, deoxythymidine, deoxyguanosine, deoxycytidine, inosine, diamino purine; base analogs, such as 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, 4-((3-(2-(2-(3-aminopropoxy)ethoxy)ethoxy)propyl)amino)pyrimidin-2(1H)-one, 4-amino-5-(hepta-1,5-diyn-1-yl)pyrimidin-2(1H)-one, 6-methyl-3,7-dihydro-2H-pyrrolo[2,3-d]pyrimidin-2-one, 3H-benzo[b]pyrimido[4,5-e][1,4]oxazin-2(10H)-one, and 2-thiocytidine; modified nucleotides, such as 2′-substituted nucleotides, including 2′-O-methylated bases and 2′-fluoro bases; and modified sugars, such as 2′-fluororibose, ribose, 2′-deoxyribose, arabinose, and hexose; and/or modified phosphate groups, such as phosphorothioates and 5′-N-phosphoramidite linkages. It is understood that an oligonucleotide is a polymer of nucleotides. In certain embodiments, the coding region does not have to contain contiguous bases. In certain embodiments, the coding region can be interspersed with linker moieties or non-nucleotide molecules.

In some embodiments, the coding region of the linear initiator nucleic acid contains from about 5% to 100%, including from about 5% to about 50%, about 40% to about 80%, about 80% to 99%, about 90% to about 99%, or about 100% DNA nucleotides. In some embodiments, the coding region contains from about 5% to about 100%, including from about 5% to about 50%, about 40% to about 80%, about 80% to about 99%, about 90% to about 99%, or about 100% RNA nucleotides. In some embodiments, wherein the coding region comprises a specified percentage of DNA nucleotides or RNA nucleotides, respectively, the remaining percentage comprises RNA nucleotides of DNA nucleotides, respectively.

In some embodiments, the linear initiator nucleic acid may further comprise a non-coding region or a plurality of non-coding regions. The term “non-coding region,” when present, refers to a region of the linear initiator nucleic acid that does not correspond to any anti-coding nucleic acid used to synthesize a compound from the linear initiator nucleic acid. In some embodiments, non-coding regions are optional. In some embodiments, the linear initiator nucleic acid contains from 1 to about 20 non-coding regions, including from 2 to about 9 non-coding regions, including from 2 to about 4 non-coding regions. In some embodiments, the non-coding regions contain from about 4 to about 50 nucleotides, including from about 12 to about 40 nucleotides, and including from about 8 to about 30 nucleotides. In some embodiments, one or more of the non-coding regions are double stranded, which reduces cross-hybridization.

The addition of non-coding regions can separate codons in the coding region to avoid or reduce cross-hybridization, because cross-hybridization would interfere with accurate encoding of a compound synthesized from the linear initiator nucleic acid. Further, the non-coding regions can add functionality to the coding region of the linear initiator nucleic acid other than just hybridization with anti-codons or encoding. The non-coding regions may be interspersed with the codons of the coding region. For example, two codons of the coding region may be separated by a non-coding region. Thus, in some embodiments, a coding region comprises one or more non-coding regions. In some embodiments, one or more of the non-coding regions can be modified with a label, such as a fluorescent label or a radioactive label. Such labels can facilitate the visualization or quantification of the linear nucleic acid. In some embodiments, one or more of the non-coding regions are modified with a functional group or tether which facilitates processing. In some embodiments, one or more of the non-coding regions are double stranded (e.g., “blocked”), which reduces cross-hybridization. Suitable non-coding regions are typically selected that do not interfere with PCR amplification of the nucleic acid portion of the linear initiator nucleic acid (e.g., non-coding regions do not interfere with identification of the building blocks used to synthesize a compound).

### Cleavable Linker

Cleavable linkers for use in the methods described herein are linkers that are capable of being cleaved. For example, in a circularized nucleic acid, cleavage of the cleavable linker causes the circularized nucleic acid to be linearized thus forming a linear initiator nucleic acid described herein.

In some embodiments, the cleavable linker comprises nucleic acids. The cleavable linker may be of any suitable length, including, in some embodiments, between about 3 and about 30 nucleotides, such as any of about 3 to about 10, about 8 to about 20, and about 15 to about 20 nucleotides. In some embodiments, the cleavable linker comprises modified nucleic acids. In some embodiments, the cleavable linker does not comprise nucleic acids. In some embodiments, the cleavable linker is a restriction site comprising a recognition sequence, wherein the recognition sequence is recognized by a restriction enzyme that cleaves the cleavable linker.

In some embodiments, the cleavable linker may be a bond that is a substrate for chemical cleavage. Thus, in some embodiments, the cleavable linker is a chemically cleavable linker (for example, olefins which can be cleaved by ruthenium catalysts). In some embodiments, the chemically cleavable linker comprises a non-nucleotide moiety. Non-nucleotide moieties which can function as cleavable linkers include, for example, disulfide bonds, photocleavable linkers, carbamoylethyl sulfones (which are typically cleaved by base), and diols (which are typically cleaved by sodium periodate). See, also, Gartner et al., Multistep Small-Molecule Synthesis Programmed by DNA Templates, J. Am. Chem. Soc. 2002, 124, 35, 10304-06. In some embodiments, the chemically cleavable linker may be acid cleavable, cleavable by reducible disulfides, or cleavable by any other suitable chemistry known in the art. A cleavable linker may be selected based on the ability of a particular type of chemical cleavage to specifically and precisely cleave a circularized nucleic acid only at the intended target site of the cleavable linker.

In some embodiments, the cleavable linker is an intervening sequence. As used herein, an “intervening sequence” is an oligonucleotide sequence that is located between a first initial building block and a second initial building block. In some embodiments, the intervening sequence is between about 5 to about 60 nucleotides long, such as any of between about 5 to about 20, about 8 to about 30, about 25 to about 40, and about 30 to about 60 nucleotides long. In some embodiments, the intervening sequence is about 5, 10, 15, 20, 25, 30, 35, 40, 45, 50, 55, or 60 nucleotides long.

### Cleavage Methods

The cleavable linker may be cleaved in the methods described herein in order to produce a linear initiator nucleic acid. Cleavage of the circularized nucleic acid produces a linear initiator nucleic acid with a first initial building block on one end, and a second initiator building block on the opposite end. An exemplary linear initiator nucleic acid is illustrated in FIG. 1. The major classes of DNA or DNA/RNA heteroduplex cleavage methods include hydrolytic cleavage (e.g., cleavage at the phosphodiester bond) and oxidative cleavage (e.g., cleavage at the sugar or base).

In some embodiments, the cleavage is enzymatic. In some embodiments, the cleavage of the cleavable linker is by restriction digestion. An exemplary circularized nucleic acid with cleavable linker and attached restriction enzyme is illustrated in FIG. 5. Restriction digestion involves the process of cleaving nucleic acid molecules (e.g., DNA or RNA) into smaller pieces at specific sequences, e.g., recognition sequences. The restriction digestion comprises cleaving nucleic acid molecules with specific enzymes (e.g., restriction enzymes) at a restriction site comprising the recognition sequence. In some embodiments, the recognition sequence comprises about 3 to about 20 nucleic acids in length, such as any of about 3 to about 6, about 5 to about 8, about 8 to about 12, or about 10 to about 20 nucleotides in length. In some embodiments, the recognition sequence comprises about 3, 4, 5, 6, 7, 8, 9, 10, 11, 12, 13, 14, 15, 16, 17, 18, 19, or 20 nucleotides in length. In some embodiments, the recognition sequence comprises the same nucleotide sequence in the 3′ to 5′ direction as the sequence in the 5′ to 3′ direction (e.g., palindromic).

There are numerous classes/types of restriction enzymes that are applicable to the methods described herein. In order to cleave nucleic acids (e.g., DNA), restriction enzymes make two incisions, once through each sugar-phosphate backbone (i.e., each strand) of the double helix. In some embodiments, the restriction enzyme cleaves a DNA double helix at a restriction site comprising a recognition sequence. In some embodiments, the restriction enzyme cleaves a DNA/RNA heteroduplex at a restriction site comprising a recognition sequence. In some embodiments, the different types of restriction enzymes cleave nucleic acids differently. For example, one restriction enzyme may cleave a 3 base pair sequence, whereas another restriction enzyme may cleave an 8 base pair sequence. In some embodiments, the cleavage conditions for restriction digestion (e.g., buffer solutions, pH, temperature, and other factors) are determined based on the distinct properties of the restriction enzyme being used. In some embodiments, the restriction enzyme is a Type I, Type II, Type III, Type IV, Type V, or an artificial restriction enzyme. In some embodiments, the restriction enzyme can be, but is not limited to, one of EcoRI, EcoRII, BamHI, HindIII, TaqI, NotI, HinF, Sau3AI, PvuII, SmaI, HaeIII, HgaI, AluI, EcoRV, EcoP15I, KpnI, PstI, SacI, SalI, ScaI, SpeI, SphI, StuI, or XbaI.

In some embodiments, the cleavable linker comprises a restriction site comprising a recognition sequence that may be targeted (e.g., cleaved) by restriction enzymes during restriction digestion. In some embodiments, the cleavable linker on the circularized nucleic acid is cleaved by restriction digestion to produce a linear initiator nucleic acid.

In some embodiments, the cleavable linker on the circularized nucleic acid is cleaved by non-enzymatic cleavage. In some embodiments, the cleavage is chemical cleavage. Chemical cleavage is based on specific cleavage of nucleic acids at a site of modification. In some embodiments, the chemical cleavage may include, but is not limited to, cleavage with dimethyl sulfate, formamidopyrimidine-DNA glycosylase (Fpg), uracil DNA glycosylase (UDG) cleavage and cleavage of carbamoyl sulfone. In some embodiments, the chemical cleavage comprises cleavage of a diol by sodium periodate. In some embodiments, the chemical cleavage is cleavage of an olefin by ruthenium catalysts.

In some embodiments, the degree of cleavage may be evaluated by gel electrophoresis or any other suitable technique known in the art.

### Methods of Synthesizing a Compound

The methods provided herein relate, in some aspects, to synthesizing a compound from a linear initiator nucleic acid (i.e., combinatorial chemistry). The compound comprises at least one polymer building block attached to a first initial building block or a second initial building block. In some embodiments, the compound comprises a plurality of polymer building blocks extending from a first initial building block or a second initial building block. The synthesized compound is connected to the linear initiator nucleic acid, which both encoded the synthesis of the synthesized compound, but also identifies the synthesized compound. Additional exemplary methods of synthesizing a compound are disclosed in US 2019/0169607 A1 and US 2020/0263163 A1, all of which may be applied to the synthesis of a compound from a linear initiator nucleic acid as described herein.

As exemplified in FIG. 1, the linear initiator nucleic acid comprises a coding region comprised of a plurality of codons, wherein the coding region corresponds to and can be used to identify the sequence of building blocks in the compound synthesized from, and encoded by, the linear initiator nucleic acid. The encoded compounds synthesized from the linear initiator nucleic acid provided herein are useful in the field of combinatorial chemistry. The synthesis method allows for the synthesis of libraries of bivalent or polyvalent molecules on a large scale directed by pools of linear initiator nucleic acid. The library of compounds can then be tested to ascertain which of them possesses the desired characteristics for the chosen application, such as binding to a target. An advantage of the present invention is allowing for the bivalent display of a synthesized compound on a single molecule. In other words, the linear initiator nucleic acid can encode for the synthesis of at least two compounds: one comprising the first initial building block and one or more polymer building blocks extending therefrom, and one comprising the second initial building block and one or more polymer building blocks extending therefrom. Having two synthesized compounds on one molecule can produce an avidity effect, which enhances binding of the molecule (i.e., the linear initiator nucleic acid bound to at least two synthesized compounds) to a target molecule.

The diagram in FIG. 7 exemplifies a method of the present invention involving sequential steps of a method of synthesizing a compound from a linear initiator nucleic acid. In some embodiments, a pool of molecules comprising a plurality of linear initiator nucleic acids are provided. In some embodiments, the linear initiator nucleic acids are synthesized by any of the methods described herein. In some embodiments, the first initial building block is on the opposite end of the linear initiator nucleic acid compared to the second initial building block. In some embodiments, the first initial building block is at the 5′ end of the linear initiator nucleic acid. In some embodiments, the second initial building block is at the 5′ end of the linear initiator nucleic acid. In some embodiments, the first initial building block is at the 3′ end of the linear initiator nucleic acid. In some embodiments, the second initial building block is at the 3′ end of the linear initiator nucleic acid.

In some embodiments, the linear initiator nucleic acid used in the synthesis of a compound comprises a first initial building block, a second initial building block, a cleavable linker, and a coding region. In some embodiments, the linear initiator nucleic acid comprises at the 5′ end a first portion of a cleavable linker and at the 3′ end a second portion of a cleavable linker. In some embodiments, the cleavable linker is an intervening sequence.

In some embodiments, the linear initiator nucleic acid used in the synthesis of a compound comprises a first initial building block, a second initial building block, an intervening sequence, and a coding region. In some embodiments, the first initial building block and/or the second initial building block is attached to the linear initiator nucleic acid via a linker. In some embodiments, the linear initiator nucleic acid comprises at the 5′ end a first portion of an intervening sequence and at the 3′ end a second portion of an intervening sequence. In some embodiments, the linear initiator nucleic acid used in the synthesis of a compound is formed from a circularized nucleic acid. In some embodiments, the circularized nucleic acid comprises a restriction site (e.g., comprising a recognition sequence) that is subjected to restriction digestion (e.g., by a restriction enzyme) to form the linear initiator nucleic acid. In some embodiments, the circularized nucleic acid does not comprise a restriction site, and is instead subjected to chemical cleavage to form the linear initiator nucleic acid. In some embodiments, the circularized nucleic acid comprises the first initial building block, the intervening sequence (e.g., comprising the restriction site or chemical cleavage site), the second initial building block, and a coding region. In some embodiments, the first initial building block and the second initial building block are attached to opposite ends of the intervening sequence in the circularized nucleic acid.

In some embodiments, the circularized nucleic acid used to form the linear initiator nucleic acid for the synthesis of a compound is formed from a linear precursor nucleic acid. In some embodiments, the linear precursor nucleic acid comprises the first initial building block, the intervening sequence (e.g., comprising the restriction site or chemical cleavage site), the second initial building block, and the coding region. In some embodiments, the first initial building block and the second initial building block are attached to opposite ends of the intervening sequence in the linear precursor nucleic acid, and are each upstream or each downstream of the coding region in the linear precursor nucleic acid. In some embodiments, the 5′ and 3′ termini of the linear precursor nucleic acid are ligated (e.g., by splint ligation) to form a circularized nucleic acid.

In some embodiments, the linear initiator nucleic acid used in the synthesis of a compound comprises a first initial building block, a second initial building block, a cleavable linker, and a coding region. In some embodiments, the first initial building block and/or the second initial building block is attached to the linear initiator nucleic acid via a linker. In some embodiments, the linear initiator nucleic acid comprises at the 5′ end a first portion of a cleavable linker and at the 3′ end a second portion of a cleavable linker. In some embodiments, the linear initiator nucleic acid used in the synthesis of a compound is formed from a circularized nucleic acid. In some embodiments, the circularized nucleic acid comprises a restriction site (e.g., comprising a recognition sequence) that is subjected to restriction digestion (e.g., by a restriction enzyme) to form the linear initiator nucleic acid. In some embodiments, the circularized nucleic acid does not comprise a restriction site, and is instead subjected to chemical cleavage to form the linear initiator nucleic acid. In some embodiments, the circularized nucleic acid comprises the first initial building block, the cleavable linker (e.g., comprising the restriction site or chemical cleavage site), the second initial building block, and a coding region. In some embodiments, the first initial building block and the second initial building block are attached to opposite ends of the cleavable linker in the circularized nucleic acid.

In some embodiments, the circularized nucleic acid used to form the linear initiator nucleic acid used in the synthesis of a compound is formed from a linear precursor nucleic acid. In some embodiments, the linear precursor nucleic acid comprises the first initial building block, the cleavable linker (e.g., comprising the restriction site or chemical cleavage site), the second initial building block, and the coding region. In some embodiments, the first initial building block and the second initial building block are attached to opposite ends of the cleavable linker in the linear precursor nucleic acid, and are each upstream or each downstream of the coding region in the linear precursor nucleic acid. In some embodiments, the 5′ and 3′ termini of the linear precursor nucleic acid are ligated (e.g., by splint ligation) to form a circularized nucleic acid.

At least one of the linear initiator nucleic acids may be contacted with a charged anti-codon. In some embodiments, the charged anti-codon comprises a polymer building block and an anti-codon that corresponds to and identifies the polymer building block of said charged anti-codon. In some embodiments, the anti-codon of the charged anti-codon is complementary to at least one of the codons of the coding region of the linear initiator nucleic acid, and when conditions permit, the anti-codon hybridizes with at least one of the codons of the coding region (FIG. 7). The polymer building block of the charged anti-codon may react with the first initial building block or the second initial building block to form a covalent bond. In some embodiments, the polymer building block may be any of, but is not limited to, the exemplary building blocks listed in the “Building blocks” section. In some embodiments, the polymer building block is not a nucleic acid or nucleic acid analog.

A second polymer building block may be attached to the unreacted first initial building block or the unreacted second initial building block. Additionally, in some embodiments, a second polymer building block may be attached to the first polymer building block, that has reacted with the first initial building block or the second initial building block. In some embodiments, the second polymer building block is identical to the first polymer building block. In some embodiments, the second polymer building block is different from the first polymer building block. In some embodiments, prior to the addition of a second charged anti-codon, the first anti-codon (e.g., the anti-codon of the first charged anti-codon) is removed from the linear initiator nucleic acid. In some embodiments, the removing of the first anti-codon comprises unhybridizing the first anti-codon from a codon of the linear initiator nucleic acid. In some embodiments, the removing of the first anti-codon comprises cleaving the first anti-codon. In some embodiments, the first anti-codon is cleaved by enzymatic digestion, such as restriction digestion or by uracil-DNA glycosylase (UDG). For example, the first anti-codon may comprise dU bases to facilitate the removal of the first anti-codon from the linear initiator nucleic acid. In some embodiments, the dU bases of the first anti-codon are cleaved by UDG. In some embodiments, the shorter fragments of the cleaved first anti-codon have lower melting temperatures, and thus lower affinity for the codon of the linear initiator nucleic acid. Thus, the cleaved first anti-codon may be outcompeted by the incoming, full-length second charged anti-codon for hybridization to the linear initiator nucleic acid, wherein the second charged anti-codon comprises an anti-codon that hybridizes to the same codon of the linear initiator nucleic acid as the anti-codon of the first charged anti-codon.

For example, in some embodiments, the linear initiator nucleic acid is contacted with a second charged anti-codon comprising a second polymer building block and a second anti-codon, that corresponds to and identifies the second polymer building block. In some embodiments, the first anti-codon is removed from the linear initiator nucleic acid (e.g., by enzymatic digestion, such as UDG cleavage, as described above) prior to the contacting of the linear initiator nucleic acid with a second anti-codon. In some embodiments, the second anti-codon hybridizes to at least one of the codons of the coding region of the linear initiator nucleic acid. In some examples, the second charged anti-codon comprises an identical polymer building block to the polymer building block of first charged anti-codon, and an anti-codon that is identical to the anti-codon of the first charged anti-codon. In some embodiments, the second anti-codon hybridizes to the same codon of the coding region of the linear initiator nucleic acid and the first anti-codon. In some embodiments, the second anti-codon hybridizes to a different codon of the coding region of the linear initiator nucleic acid and the first anti-codon. In some embodiments, the second polymer building block of the second charged anti-codon reacts with the first polymer building block to form a covalent bond. In some embodiments, the second polymer building block of the second charged anti-codon reacts with the unreacted first initial building block or with the unreacted second initial building block to form a covalent bond.

The method of contacting a linear initiator nucleic acid with a charged anti-codon comprising a polymer building block and an anti-codon, hybridizing the anti-codon with at least one of the codons of the coding region of the linear initiator nucleic acid, and reacting the polymer building block with the building block previously attached to the linear initiator nucleic acid to form a covalent bond, may be repeated multiple times. In some embodiments, the method is repeated 3, 4, 5, 6, 7, 8, 9, or 10 times. In some embodiments, the method forms a synthesized compound comprising a plurality of polymer building blocks extending from the first initial building block and a synthesized compound comprising a plurality of polymer building blocks extending from the second initial building block.

In some embodiments, the synthesized compounds comprising a first initial building block or a second initial building block are attached to the nucleic acid portion (e.g., the coding region, non-coding region, and/or the cleavable linker or intervening sequence) of the linear initiator nucleic acid via a linker.

The nucleic acid portion of the linear initiator nucleic acid may be designed to not interfere with the functionality of the synthesized compound. In some embodiments, the synthesis of the compound occurs under conditions that is compatible with the nucleic acid portion of the linear initiator nucleic acid, to reduce the loss of the coding information. For example, extreme reaction conditions such as prolonged reaction at high temperatures, acidic environments, oxidants, and transition-metal ions may degrade the nucleic acid portion of the linear initiator nucleic acid.

In some embodiments, the synthesized compound comprising the first initial building block and the synthesized compound comprising the second initial building block are different. In some embodiments, the synthesized compound comprising the first initial building block and the synthesized compound comprising the second initial building block are the same. In some embodiments, the synthesized compounds comprise polymer building blocks that may be any of, but are not limited to, the exemplary building blocks listed in the “Building blocks” section. In some embodiments, the synthesized compounds, comprising either the first initial building block or the second initial building block, do not comprise a nucleic acid or nucleic acid analog.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1. Synthesis of a Linear Initiator Nucleic Acid

This example demonstrates the synthesis of a linear initiator nucleic acid. In particular, this example demonstrates the synthesis of a linear initiator nucleic acid from a linear precursor nucleic acid and a circularized nucleic acid.

FIG. 6 illustrates an exemplary method of making a linear initiator nucleic acid from a linear precursor nucleic acid starting material. A linear precursor nucleic acid comprising a first initial building block and a second initial building block is circularized by splint ligation or blunt end ligation. A cleavable linker positioned at a site in between the site of the first initial building block and the site of the second initial building block is then cleaved, either by restriction digestion or chemical cleavage. Cleavage of the circularized nucleic acid results in a linear initiator nucleic acid wherein the first initial building block and the second initial building block are moved to sites near opposite ends of the nucleic acid.

A pool of linear initiator nucleic acids is then used for the production of combinatorial chemical libraries, to encode and direct the synthesis of compounds extending from the first initial building block and from the second initial building block.

### Example 2. Synthesis of a Compound

This example demonstrates the synthesis of a compound from a linear initiator nucleic acid, as illustrated in an exemplary diagram shown in FIG. 7.

A pool of linear initiator nucleic acids is provided, wherein the linear initiator nucleic acids comprise a first initial building block, a second initial block, and a coding region. As exemplified in FIG. 7, at least one charged anti-codon (an anti-codon carrying a polymer building block, e.g., a first charged anti-codon) may hybridize with at least one codon of the plurality of codons within the coding region of the linear initiator nucleic acid. The first charged anti-codon comprises a polymer building block and an anti-codon that is complementary to a codon of the linear initiator nucleic acid. The polymer building block reacts with and covalently bonds to the first initial building block or the second initial building block.

A second, optionally identical, polymer building block may be attached to the unreacted first initial building block or the unreacted second initial building block. The first charged anti-codon is removed (e.g., cleaved and unhybridized) from the linear initiator nucleic acid, and a second charged anti-codon comprising a second copy of the polymer building block and an anti-codon that is complementary to a codon of the linear initiator nucleic acid, may hybridize with at least one codon of the plurality of codons within the coding region of the linear initiator nucleic acid. In some examples, the second charged anti-codon comprises an identical polymer building block to the polymer building block of first charged anti-codon, and an anti-codon that is identical to the anti-codon of the first charged anti-codon. In these examples the anti-codon of the second charged anti-codon is complementary to the same codon of the linear initiator nucleic acid as the anti-codon of the first charged anti-codon. The second copy of the polymer building block on the second charged anti-codon reacts with and covalently bonds to the unreacted second initial building block.

Optionally, various additional anti-codons comprising different or the same polymer building blocks may hybridize to a codon of the coding region of the linear initiator nucleic acid. Such a method may ultimately form a synthesized compound comprising a plurality of polymer building blocks extending from the first initial building block and a synthesized compound comprising a plurality of polymer building blocks extending from the second initial building block. In some examples, as shown in FIG. 8, the synthesized compound comprising the first initial building block and the synthesized compound comprising the second initial building block are the same. Alternatively, the synthesized compound comprising the first initial building block and the synthesized compound comprising the second initial building block may be different.

The synthesized compound corresponds to and may be identified by the coding region of the linear initiator nucleic acid. Compounds may be subjected to downstream analysis for selection of compounds possessing specific properties (e.g., binding to a particular target molecule). The coding region of the compounds selected for said properties can be PCR amplified to determine the identity of the building blocks.

1. A method of making a linear initiator nucleic acid,
wherein the linear initiator nucleic acid comprises a first initial building block, a second initial building block, and a coding region; and
wherein the first initial building block is attached to a first site that is upstream of the coding region on the linear initiator nucleic acid and the second initial building block is attached to a second site that is downstream of the coding region on the linear initiator nucleic acid;
the method comprising cleavage of a circularized nucleic acid to form the linear initiator nucleic acid;
wherein the circularized nucleic acid comprises (i) the first initial building block, (ii) a cleavable linker, (iii) the second initial building block, and (iv) the coding region; and
wherein (i) and (iii) are attached to opposite ends of (ii), and wherein the cleavage cleaves the cleavable linker.

2. The method of claim 1, wherein cleavage is by enzymatic cleavage or by chemical cleavage.

3-4. (canceled)

5. The method of claim 1, wherein the circularized nucleic acid is formed by a method comprising ligation of a linear precursor nucleic acid to form the circularized nucleic acid; wherein the linear precursor nucleic acid comprises (i) the first initial building block, (ii) the cleavable linker, (iii) the second initial building block, and (iv) the coding region;
wherein (i) and (iii) are attached to opposite ends of (ii) in the linear precursor nucleic acid; and
wherein (i), (ii), and (iii) are each upstream or each downstream of (iv) in the linear precursor nucleic acid.

6-7. (canceled)

8. The method of claim 1, wherein the coding region comprises a plurality of codons.

9. (canceled)

10. The method of claim 8, wherein at least one codon encodes the addition of a polymer building block to the first initial building block, the second initial building block, or both.

11. The method of claim 8, wherein the plurality of codons encodes for the addition of a plurality of polymer building blocks.

12. The method of claim 1, wherein the linear initiator nucleic acid comprises a first linker and a second linker, wherein the first linker attaches the first initial building block to the linear initiator nucleic acid, and the second linker attaches the second initial building block to the linear initiator nucleic acid.

13. (canceled)

14. The method of claim 1, wherein the first initial building block and the second initial building block are not nucleic acids or nucleic acid analogs.

15-16. (canceled)

17. The method of claim 1, wherein the cleavable linker is an intervening sequence.

18. The method of claim 17, wherein:
the intervening sequence is from 4 to 30 nucleotides long; or
the intervening sequence is a non-nucleotide moiety.

19. (canceled)

20. A linear precursor nucleic acid comprising (i) a first initial building block, (ii) a cleavable linker, (iii) a second initial building block, and (iv) a coding region;
wherein (i) and (iii) are attached to opposite ends of (ii) in the linear precursor oligonucleotide; and
wherein (i), (ii), and (iii) are each upstream or each downstream of (iv) in the linear precursor nucleic acid.

21. The precursor nucleic acid of claim 20, wherein the 5′ and 3′ termini are non-covalently bound to a nucleotide splint.

22. A circularized nucleic acid comprising (i) a first initial building block, (ii) a cleavable linker, (iii) a second initial building block, and (iv) a coding region;
wherein (i) and (iii) are attached to opposite ends of (ii).

23. A method of synthesizing a compound comprising:
(a) providing a pool of molecules comprising a plurality of linear initiator nucleic acids, wherein each linear initiator nucleic acid comprises a first initial building block, a second initial building block, and a coding region comprising a plurality of codons; wherein the first initial building block is attached to a site that is upstream of the coding region on the linear initiator nucleic acid and the second initial building block is attached to a second site that is downstream of the coding region on the linear initiator nucleic acid; and
(b) contacting at least one of the linear initiator nucleic acids with an anti-codon comprising a polymer building block under conditions which allow for hybridization of the anti-codon with at least one of the codons of the coding region, wherein the polymer building block reacts with the first initial building block or the second initial building block to form a covalent bond.

24. The method of claim 23, wherein the linear initiator nucleic acid is formed by cleavage of a cleavable linker at a cleavage site in a circularized nucleic acid comprising (i) the first initial building block, (ii) the cleavable linker comprising the cleavage site, (iii) a second initial building block, and (iv) the coding region, and wherein (i) and (iii) are attached to opposite ends of (ii) in the circularized nucleic acid.

25. The method of claim 23, wherein:
(a) the linear initiator nucleic acid comprises at the 5′ end a first portion of an intervening sequence and at the 3′ end a second portion of an intervening sequence;
wherein the linear initiator nucleic acid was formed by restriction digestion of a restriction site in a circularized nucleic acid comprising (i) the first initial building block, (ii) the intervening sequence comprising the restriction site, (iii) a second initial building block, and (iv) the coding region; and
wherein (i) and (iii) are attached to opposite ends of (ii) in the circularized nucleic acid, or
(b) the linear initiator nucleic acid comprises at the 5′ end a first portion of a cleavable linker and at the 3′ end a second portion of a cleavable linker;
wherein the linear initiator nucleic acid was formed by restriction digestion of a restriction site in a circularized nucleic acid comprising (i) the first initial building block, (ii) the cleavable linker comprising the restriction site, (iii) a second initial building block, and (iv) the coding region; and
wherein (i) and (iii) are attached to opposite ends of (ii) in the circularized nucleic acid.

26-27. (canceled)

28. The method of claim 23, further comprising repeating step (b) to form a synthesized compound comprising a plurality of polymer building blocks extending from the first initial building block and a synthesized compound comprising a plurality of polymer building blocks extending from the second initial building block.

29. The method of claim 28, wherein the synthesized compound comprising the first initial building block and the synthesized compound comprising the second initial building block are the same.

30. The method of claim 23, wherein the polymer building block is not a nucleic acid or nucleic acid analog.

31. The method of claim 28, wherein the synthesized compound does not comprise a nucleic acid or nucleic acid analog.
